(19) United States
(12) Patent Application Publication
(10) Pub. No.: US 2007/0066833 A1
(43) Pub. Date: Mar. 22, 2007

(54) Methods to synthesize indoles as precursors for pharmaceuticals

(75) Inventors: Sundeep Dugar, San Jose, CA (US); Peter Giannousis, Pacifica, CA (US)

Correspondence Address: MORRISON & FOERSTER LLP, 12531 HIGH BLUFF DRIVE, SUITE 100, SAN DIEGO, CA, 92130-2040, US

(73) Assignee: Scios, Inc., Fremont, CA (US)

(21) Appl. No.: 11/507,869

(22) Filed: Aug. 21, 2006

Related U.S. Application Data
(60) Provisional application No. 60/709,652, filed on Aug. 19, 2005.

(51) Int. Cl. C07D 209/04

(52) U.S. Cl. 548/508

(57) ABSTRACT

The invention provides improved methods for the synthesis of indoles, especially useful for making indoles having an ester or amide group at C-5. The methods involve cyclization of a protected ortho-acetylene-substituted aniline derivative to produce an indole.

### CROSS-REFERENCE TO RELATED APPLICATIONS

This application claims priority to U.S. provisional application 60/709,652 filed Aug. 19, 2005. The contents of that provisional application are incorporated herein by reference.

### TECHNICAL FIELD

This invention relates to methods for synthesizing certain indole compounds that are useful as intermediates for preparation of pharmaceuticals. It provides improved methods to synthesize intermediates that can be used to manufacture pharmaceuticals, including those described in U.S. Pat. No. 6,867,209 and related patents.

### BACKGROUND ART

Many known pharmaceuticals and agrochemicals contain an indole ring system, and numerous synthetic methods are available for making indoles having various substitution patterns. U.S. Pat. No. 6,867,209 and U.S. Pat. No. 6,890,938, for example, disclose p38 inhibitors that contain an indole or similar ring system, and also contain an acyl group and an additional aryl group. Several methods to prepare such indole compounds are described in those patents. One of the disclosed methods involves the synthesis of iodinated anilines that can be transformed into indoles via a Sonogashira acetylenation followed by cyclization as depicted in Scheme I.

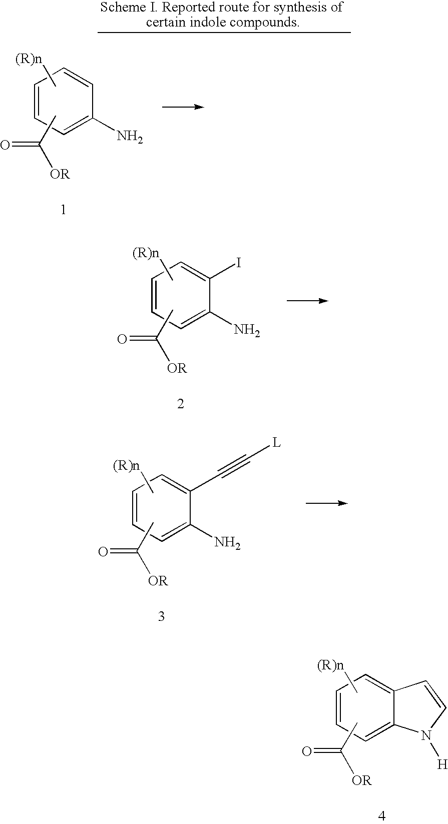

Similar methods for constructing an indole from an aniline have also been reported elsewhere. See e.g. G. Kursch, et al., Current Org. Synthesis, 1, 47-63 (2004). However, the yield for the acetylene attachment is often not satisfactory, which is especially important when a relatively expensive acetylene like trimethylsilyl acetylene is used. In some cases it has been found that no cyclized product is obtained from compounds of formula 3. While it could be helpful to protect the amine during this reaction, use of a protecting group typically requires the addition of two steps rather than one, because it also becomes necessary to de-protect the amine after the reaction. Therefore, there is a need for improved methods to synthesize these compounds. The present invention provides such improved methods, including methods that are especially useful for large-scale manufacturing.

### DISCLOSURE OF THE INVENTION

The invention provides improved methods for synthesizing certain intermediates that are useful for preparation of indole-containing pharmaceutical compounds. The invention provides novel intermediates and methods for making indole compounds of formula 5 or 6 from para-aminobenzoate derivatives, and is especially useful for preparation of compounds of formula 6:

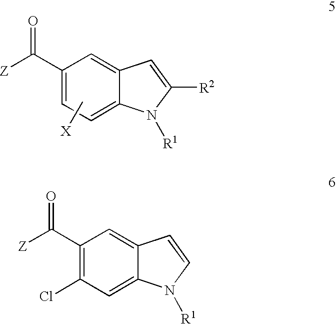

wherein R¹ is H or an alkyl, acyl, alkoxycarbonyl, or alkylsulfonyl;

R² is H or an alkyl, aryl, arylalkyl or silyl group;

X represents 0-3 substituents;

Y is H, optionally substituted C1-C6 alkyl, optionally substituted C5-C12 aryl including heteroaryl groups containing 1-3 heteroatoms selected from N, O and S, optionally substituted C6-C13 arylalkyl, an optionally substituted C1-C6 alkylsulfonyl group, an optionally substituted C5-C12 arylsulfonyl group, an optionally substituted C1-C6 alkoxycarbonyl group, or an optionally substituted C1-C6 acyl group; and

Z is OR or NR₂, or Z may be an optionally substituted 5-6 membered azacyclic group that forms an amide with the benzoate carbonyl;

wherein each R is independently H or optionally substituted C1-C6 alkyl.

The indole compounds prepared by methods of the invention may also be isolated as salts, and may be further functionalized using known methods.

The methods of the invention involve use of a protecting group on the amine portion of an aniline as depicted in Scheme II. Depending on the protecting group (PG) used and the conditions used for the cyclization, the product of formula (III) may have either PG or H at N-1 of the newly-formed indole. Also depending on the cyclization conditions and on the nature of L, the indole of formula (III) may contain either L or H at C-2.

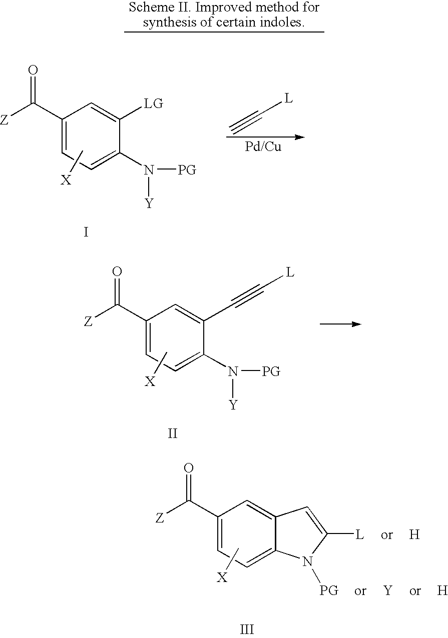

wherein:
LG is a leaving group;
PG is a protecting group;
Y is H or optionally substituted C1-C6 alkyl or optionally substituted C1-C6 sulfonyl, or Y can be a protecting group that may be the same as or different from PG;
Z is OR or NR₂ or a 5-6 membered azacyclic group that forms an amide with the benzoate carbonyl;
X represents 0-3 substituents selected from halo, CF₃, R, and OR;
L is an optionally substituted C1-C6 alkyl group or R₃Si;
and each R is independently C1-C6 alkyl or phenyl.

Thus in one aspect, the invention provides a method to synthesize indole compounds of formula (III) from an acetylene-containing para-amino benzoate derivative of formula (II) according to Scheme II. The methods also use a para-amino benzoate derivative of formula (I) to make the acetylene-containing compound of formula (II) by using a catalyst, typically a palladium (O) or palladium (II) catalyst. The compound of formula (II) is cyclized to form the product of formula (III). If L is a silyl group, the cyclization may be accomplished using fluoride ion in an organic solvent, in which case the product is an indole of formula (III) having H at C-2 of the indole.

The methods of the invention employ a palladium-catalyzed acetylenation reaction that introduces a substituted acetylene such as trimethylsilyl acetylene, which is a relatively expensive material; thus improvements in its introduction and in subsequent steps can substantially reduce the cost to manufacture the desired indole intermediates. The protecting group on the aniline nitrogen in a compound of formula (II) improves the overall efficiency of the reaction sequence, because it improves the yields of both the acetylene incorporation reaction and the subsequent cyclization reaction. Indeed in some cases, especially where L in a compound of formula II is a trimethylsilyl group, if no protecting group is used on the amino group (i.e., where PG is H), the cyclization reaction completely fails under fluoride-induced conditions: the isolated product is not an indole at all, but a desilylated version of the acetylenic starting material (i.e., a compound of formula (II) wherein L is H).

### MODES OF CARRYING OUT THE INVENTION

As used herein, the terms ‘ortho’ and ‘meta’ and ‘para’ refer to the relative positions of substituents on an aromatic ring, typically a phenyl ring. These terms are used in their ordinary chemical sense, whereby ‘ortho’ describes groups in a 1,2 relative orientation on the aromatic ring; ‘meta’ describes groups in a 1,3 relative orientation; and ‘para’ describes groups in a 1,4-relative orientation. Thus ‘ortho’ means the named substituent is on a ring carbon adjacent to that occupied by a reference substituent. For example, “ortho-LG aniline” refers to an aniline (aminobenzene) wherein the group LG is on the ring carbon adjacent to that occupied by the amine, regardless of the presence of other substituents.

As used herein, the terms C1-C4, C1-C6 and the like refer to groups having 1-4 or 1-6 carbon atoms respectively. The carbon atoms may be arranged in a linear, branched, or cyclic configuration or a combination of these, as long as the arrangement of atoms is consistent with chemical stability as understood in the pharmaceutical arts. A C1-C6 alkoxy group refers to a C1-C6 group that is connected through an oxygen atom.

As used herein, the terms “alkyl,” “alkenyl” and “alkynyl” include straight-chain, branched-chain and cyclic monovalent hydrocarbyl radicals, and combinations of these, which contain only C and H when they are unsubstituted. Examples include methyl, ethyl, isobutyl, cyclohexyl, cyclopentylethyl, 2-propenyl, 3-butynyl, and the like. The total number of carbon atoms in each such group is sometimes described herein, e.g., when the group can contain up to ten carbon atoms it can be represented as 1-10C or as C1-C10 or C1-C10. When heteroatoms (N, O and S typically) are allowed to replace carbon atoms as in heteroalkyl groups, for example, the numbers describing the group, though still written as e.g. C1-C6, represent the sum of the number of carbon atoms in the group plus the number of such heteroatoms that are included as replacements for carbon atoms in the ring or chain being described.

Certain alkynyl groups are referred to herein by common names such as acetylene or as acetylenic groups; these often refer to common acetylenic compounds and substituents such as trimethylsilyl acetylene and —C≡CSiMe₃, for example.

Typically, the alkyl, alkenyl and alkynyl substituents of the invention contain 1-10C (alkyl) or 2-10C (alkenyl or alkynyl) unless otherwise specified. Preferably they contain 1-8C (alkyl) or 2-8C (alkenyl or alkynyl). Sometimes they contain 1-4C (alkyl) or 2-4C (alkenyl or alkynyl). A single group can include more than one type of multiple bond, or more than one multiple bond; such groups are included within the definition of the term “alkenyl” when they contain at least one carbon-carbon double bond, and are included within the term “alkynyl” when they contain at least one carbon-carbon triple bond.

Alkyl, alkenyl and alkynyl groups are often substituted to the extent that such substitution makes sense chemically. Typical substituents include, but are not limited to, halo, ═O, ═N—CN, ═N—OR, ═NR, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C1-C8 acyl, C2-C8 heteroacyl, C2-C8 alkenyl, C2-C8 heteroalkenyl, C2-C8 alkynyl, C2-C8 heteroalkynyl, C6-C10 aryl, or C5-C10 heteroaryl, and each R is optionally substituted with halo, ═O, ═N—CN, ═N—OR′, ═NR′, OR′, NR′₂, SR′, SO₂R′, SO₂NR′₂, NR′SO₂R′, NR′CONR′₂, NR′COOR′, NR′COR′, CN, COOR′, CONR′₂, OOCR′, COR′, and NO₂, wherein each R′ is independently H, C1-C8 alkyl, C2-C8 heteroalkyl, C1-C8 acyl, C2-C8 heteroacyl, C6-C10 aryl or C5-C10 heteroaryl. Alkyl, alkenyl and alkynyl groups can also be substituted by C1-C8 acyl, C2-C8 heteroacyl, C6-C10 aryl or C5-C10 heteroaryl, each of which can be substituted by the substituents that are appropriate for the particular group.

While “alkyl” as used herein includes cycloalkyl and cycloalkylalkyl groups, the term “cycloalkyl” may be used herein to describe a carbocyclic non-aromatic group that is connected via a ring carbon atom, and “cycloalkylalkyl” may be used to describe a carbocyclic non-aromatic group that is connected to the molecule through an alkyl linker. Similarly, “heterocyclyl” may be used to describe a non-aromatic cyclic group that contains at least one heteroatom as a ring member and that is connected to the molecule via a ring atom, which may be C or N; and “heterocyclylalkyl” may be used to describe such a group that is connected to another molecule through a linker. The sizes and substituents that are suitable for the cycloalkyl, cycloalkylalkyl, heterocyclyl, and heterocyclylalkyl groups are the same as those described above for alkyl groups. As used herein, these terms also include rings that contain a double bond or two, as long as the ring is not aromatic.

As used herein, “acyl” encompasses groups comprising an alkyl, alkenyl, alkynyl, aryl or arylalkyl radical attached at one of the two available valence positions of a carbonyl carbon atom, and heteroacyl refers to the corresponding groups wherein at least one carbon other than the carbonyl carbon has been replaced by a heteroatom chosen from N, O and S. Thus heteroacyl includes, for example, —C(═O)OR and —C(═O)NR₂ as well as —C(═O)-heteroaryl.

Acyl and heteroacyl groups are bonded to any group or molecule to which they are attached through the open valence of the carbonyl carbon atom. Typically, they are C1-C8 acyl groups, which include formyl, acetyl, pivaloyl, and benzoyl, and C2-C8 heteroacyl groups, which include methoxyacetyl, ethoxycarbonyl, and 4-pyridinoyl. The hydrocarbyl groups, aryl groups, and heteroforms of such groups that comprise an acyl or heteroacyl group can be substituted with the substituents described herein as generally suitable substituents for each of the corresponding component of the acyl or heteroacyl group.

“Aromatic” moiety or “aryl” moiety refers to a monocyclic or fused bicyclic moiety having the well-known characteristics of aromaticity; examples include phenyl and naphthyl. Similarly, “heteroaromatic” and “heteroaryl” refer to such monocyclic or fused bicyclic ring systems which contain as ring members one or more heteroatoms selected from O, S and N. The inclusion of a heteroatom permits aromaticity in 5-membered rings as well as 6-membered rings. Typical heteroaromatic systems include monocyclic C5-C6 aromatic groups such as pyridyl, pyrimidyl, pyrazinyl, thienyl, furanyl, pyrrolyl, pyrazolyl, thiazolyl, oxazolyl, and imidazolyl and the fused bicyclic moieties formed by fusing one of these monocyclic groups with a phenyl ring or with any of the heteroaromatic monocyclic groups to form a C8-C10 bicyclic group such as indolyl, benzimidazolyl, indazolyl, benzotriazolyl, isoquinolyl, quinolyl, benzothiazolyl, benzofuranyl, pyrazolopyridyl, quinazolinyl, quinoxalinyl, cinnolinyl, and the like. Any monocyclic or fused ring bicyclic system which has the characteristics of aromaticity in terms of electron distribution throughout the ring system is included in this definition. It also includes bicyclic groups where at least the ring which is directly attached to the remainder of the molecule has the characteristics of aromaticity. Typically, the ring systems contain 5-12 ring member atoms. Preferably the monocyclic heteroaryls contain 5-6 ring members, and the bicyclic heteroaryls contain 8-10 ring members.

Aryl and heteroaryl moieties may be substituted with a variety of substituents including C1-C8 alkyl, C2-C8 alkenyl, C2-C8 alkynyl, C5-C12 aryl, C1-C8 acyl, and heteroforms of these, each of which can itself be further substituted; other substituents for aryl and heteroaryl moieties include halo, OR, NR₂, SR, SO₂R, SO₂NR₂, NRSO₂R, NRCONR₂, NRCOOR, NRCOR, CN, COOR, CONR₂, OOCR, COR, and NO₂, wherein each R is independently H, C1-C8 alkyl, C2-C8 heteroalkyl, C2-C8 alkenyl, C2-C8 heteroalkenyl, C2-C8 alkynyl, C2-C8 heteroalkynyl, C6-C10 aryl, C5-C10 heteroaryl, C7-C12 arylalkyl, or C6-C12 heteroarylalkyl, and each R is optionally substituted as described above for alkyl groups. The substituent groups on an aryl or heteroaryl group may of course be further substituted with the groups described herein as suitable for each type of such substituents or for each component of the substituent. Thus, for example, an arylalkyl substituent may be substituted on the aryl portion with substituents described herein as typical for aryl groups, and it may be further substituted on the alkyl portion with substituents described herein as typical or suitable for alkyl groups.

Similarly, “arylalkyl” and “heteroarylalkyl” refer to aromatic and heteroaromatic ring systems which are bonded to their attachment point through a linking group such as an alkylene, including substituted or unsubstituted, saturated or unsaturated, cyclic or acyclic linkers. Typically the linker is C1-C8 alkyl or a hetero form thereof. These linkers may also include a carbonyl group, thus making them able to provide substituents as an acyl or heteroacyl moiety. An aryl or heteroaryl ring in an arylalkyl or heteroarylalkyl group may be substituted with the same substituents described above for aryl groups. Preferably, an arylalkyl group includes a phenyl ring optionally substituted with the groups defined above for aryl groups and a C1-C4 alkylene that is unsubstituted or is substituted with one or two C1-C4 alkyl groups or heteroalkyl groups, where the alkyl or heteroalkyl groups can optionally cyclize to form a ring such as cyclopropane, dioxolane, or oxacyclopentane. Similarly, a heteroarylalkyl group preferably includes a C5-C6 monocyclic heteroaryl group that is optionally substituted with the groups described above as substituents typical on aryl groups and a C1-C4 alkylene that is unsubstituted or is substituted with one or two C1-C4 alkyl groups or heteroalkyl groups, or it includes an optionally substituted phenyl ring or C5-C6 monocyclic heteroaryl and a C1-C4 heteroalkylene that is unsubstituted or is substituted with one or two C1-C4 alkyl or heteroalkyl groups, where the alkyl or heteroalkyl groups can optionally cyclize to form a ring such as cyclopropane, dioxolane, or oxacyclopentane.

Where an arylalkyl or heteroarylalkyl group is described as optionally substituted, the substituents may be on either the alkyl or heteroalkyl portion or on the aryl or heteroaryl portion of the group. The substituents optionally present on the alkyl or heteroalkyl portion are the same as those described above for alkyl groups generally; the substituents optionally present on the aryl or heteroaryl portion are the same as those described above for aryl groups generally.

“Arylalkyl” groups as used herein are hydrocarbyl groups if they are unsubstituted, and are described by the total number of carbon atoms in the ring and alkylene or similar linker. Thus a benzyl group is a C7-arylalkyl group, and phenylethyl is a C8-arylalkyl.

In general, any alkyl, alkenyl, alkynyl, acyl, or aryl or arylalkyl group or any heteroform of one of these groups that is contained in a substituent may itself optionally be substituted by additional substituents. The nature of these substituents is similar to those recited with regard to the primary substituents themselves if the substituents are not otherwise described. Thus, where an embodiment of, for example, R⁷ is alkyl, this alkyl may optionally be substituted by the remaining substituents listed as embodiments for R⁷ where this makes chemical sense, and where this does not undermine the size limit provided for the alkyl per se; e.g., alkyl substituted by alkyl or by alkenyl would simply extend the upper limit of carbon atoms for these embodiments, and is not included. However, alkyl substituted by aryl, amino, alkoxy, ═O, and the like would be included within the scope of the invention, and the atoms of these substituent groups are not counted in the number used to describe the alkyl, alkenyl, etc. group that is being described. Where no number of substituents is specified, each such alkyl, alkenyl, alkynyl, acyl, or aryl group may be substituted with a number of substituents according to its available valences; in particular, any of these groups may be substituted with fluorine atoms at any or all of its available valences, for example.

“Optionally substituted” as used herein indicates that the particular group or groups being described may have no non-hydrogen substituents, or the group or groups may have one or more non-hydrogen substituents. If not otherwise specified, the total number of such substituents that may be present is equal to the number of H atoms present on the unsubstituted form of the group being described. Where an optional substituent is attached via a double bond, such as a carbonyl oxygen (═O), the group takes up two available valences, so the total number of substituents that may be included is reduced according to the number of available valences.

“Halo”, as used herein includes fluoro, chloro, bromo and iodo.

“Amino” as used herein refers to NH₂, but where an amino is described as “substituted” or “optionally substituted”, the term includes NR′R″ wherein each R′ and R″ is independently H, or is an alkyl, alkenyl, alkynyl, acyl, aryl, or arylalkyl group or a heteroform of one of these groups, and each of the alkyl, alkenyl, alkynyl, acyl, aryl, or arylalkyl groups or heteroforms of one of these groups is optionally substituted with the substituents described herein as suitable for the corresponding group. The term also includes forms wherein R′ and R″ are linked together to form a 3-8 membered ring which may be saturated, unsaturated or aromatic and which contains 1-3 heteroatoms independently selected from N, O and S as ring members, and which is optionally substituted with the substituents described as suitable for alkyl groups or, if NR′R″ is an aromatic group, it is optionally substituted with the substituents described as typical for heteroaryl groups.

As used herein, “azacyclic group” refers to a single ring, typically having 5-6 ring members, that is either saturated or unsaturated but is not aromatic, and that contains at least one N as a ring atom: it is attached to the benzoate carbonyl through a nitrogen atom in the azacyclic ring, so the compound is a benzamide. Typical azacyclic groups within the scope of the invention include pyrrolidines, pyrrolidinones, piperidines, morpholines, piperazines, piperazinones, and the like, each of which is optionally substituted.

As used herein, “para-aminobenzoate derivative” refers to any ester or amide formed with the carboxylic acid group of para-amino benzoic acid, and also encompasses the free carboxylic acids themselves. These derivatives may have substituents at any or all of the ring carbons of the para-aminobenzoic acid phenyl ring.

In one aspect, the invention provides intermediates for an improved synthesis of indole compounds that are useful for the preparation of pharmaceuticals. The novel intermediates of the invention are compounds of formula (I) and formula (II):

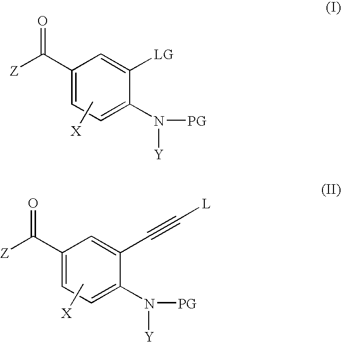

wherein:
LG is a leaving group;
PG is a protecting group;
Z is OR or NR₂ or a 5-6 membered azacyclic group that forms an amide with the benzoate carbonyl;
L is an optionally substituted C1-C6 alkyl group or R₃Si;
Y is H, optionally substituted C1-C6 alkyl, optionally substituted C5-C12 aryl including heteroaryl groups containing 1-3 heteroatoms selected from N, O and S, optionally substituted C6-C13 arylalkyl, an optionally substituted C1-C6 alkylsulfonyl group, an optionally substituted C5-C12 arylsulfonyl group, an optionally substituted C1-C6 alkoxycarbonyl group, or an optionally substituted C1-C6 acyl group;
X represents 0-3 substituents selected from halo, CF₃, C1-C6 alkyl and C1-C6 alkoxy;
and each R is independently C1-C6 alkyl or phenyl.

In another aspect, the invention provides improved methods for synthesizing certain indole compounds that are useful as intermediates for the preparation of pharmaceuticals. In one such method, which is depicted in Scheme II, the amine group of an acetylene-containing para-amino benzoate derivative of formula (II) is cyclized to form an indole of formula (III). An ortho-LG-aniline of formula (I) protected with a suitable protecting group such as an acyl group or alkoxycarbonyl group can be used to prepare the compound of formula (II). LG is then replaced by an acetylenic group, and the product, an ortho-acetylenyl aniline, is cyclized to form an indole. If Y is H in formula (II), the product of this sequence is an indole that is unsubstituted at N-1.

It is also possible to produce an N-substituted indole by the methods described herein. In compounds of formula (II), if Y is an optionally substituted alkyl group or an optionally substituted alkylsulfonyl group, the Y group will remain on the nitrogen during the cyclization. Thus, where the target compound has such a group at N-1 of the indole, that Y group can be incorporated before formation of the indole ring, such as by alkylation or alkyl- or aryl-sulfonylation of the protected aniline of formula I or alkylation or alkyl- or aryl-sulfonylation of one of its precursors.

Methods for the preparation of anilines including para-aminobenzoate derivatives having a leaving group (LG) ortho to the aniline amine are known in the art, and conditions for protection of such anilines are also well known and are discussed further below. An acetylene group can be attached to such para-amino-benzoate derivatives by replacement of a leaving group as shown in Scheme II, using Sonogashira reaction conditions where LG is iodide (—I), for example or using similar palladium-catalyzed reaction conditions where LG may be I, Br, OTf, R₃Sn—, —B(OR)₂, or other functional groups.

It has now been found that protection of the amine group of the aniline prior to the installation of the acetylenic group significantly improves the yield of the acetylene-incorporation reaction in some instances. The protecting group PG also improves the yield of the cyclization of the ortho-acetylenyl-aniline to form an indole: in fact, in some cases such as where L is a trimethylsilyl group, the fluoride-induced cyclization reaction fails if the amine group of the aniline is a free NH₂. Suitable selection of the protecting group PG and of the cyclization conditions has also been found to produce the indole without the need for a separate step to remove the protecting group. Thus the protection of the amine of the aniline can be accomplished with one high-yield step, does not necessarily require adding a separate deprotection step, and improves the efficiency of the overall process.

LG may be a halogen or other leaving group that can be replaced using Pd-catalyzed acetylenation conditions. Examples of LG include —OTf (trifluoromethanesulfonyloxy), I, and Br, although a boronate group (—B(OR)₂ where each R is H or C1-C6 alkyl, and wherein two R groups may cyclize to form a ring of up to 8 members), or —SnR₃ or —SiR₃ wherein each R is independently C1-C6 alkyl or phenyl, or a mercurial HgX, where X represents halo, may also be used; often LG is I or Br or —OTf. In some embodiments of the methods of the invention, LG is I.

PG is a protecting group suitable for use on an aniline nitrogen. Typically, the protecting group PG is an optionally substituted C1-C10 acyl group such as formyl, acetyl, methoxyacetyl, trifluoroacetyl, propionyl, benzoyl, phenylacetyl, and the like, or it may be an optionally substituted alkoxycarbonyl such as methoxycarbonyl, phenoxycarbonyl, trichloroethoxycarbonyl, benzyloxycarbonyl, t-butyloxycarbonyl, and the like. Suitable substituents for the optionally substituted acyl and alkoxycarbonyl protecting groups include halo, C5-C12-aryl, and C1-C6 alkoxy groups. In some embodiments, the protecting group is an unsubstituted C1-C6 acyl group such as formyl or acetyl.

The protecting group PG is sometimes introduced after introduction of LG. For example, where LG represents —I, the iodo group may be introduced by iodination of an aniline using conventional methods such as treatment with iodine and sodium metaperiodate in a polar solvent such as DMF. The protecting group PG may be added to the NH₂ of the ortho-iodo aniline using conventional methods.

However, other methods for synthesis of the N-protected ortho-LG aniline may be used, and other leaving groups besides I, particularly Br or triflate (—OTf), may be used as LG on the aniline. Where LG represents —OTf, for example, it may be preferable to protect the amine group of an ortho-amino-phenol first, and form the triflate of the phenol later. Methods for installing suitable protecting groups on anilines are well known in the art, and can be found for example in T. Green and Wuts, Protective Groups in Organic Synthesis, Wiley Press (1991), which is incorporated herein by reference for its disclosure of such methods. Typical methods for introducing the protecting groups include reaction of the aniline with an acyl halide or an acyl anhydride, including a mixed anhydride, usually in the presence of a proton acceptor such as a teriary amine base or a pyridine base. Acyl halides useful in the reaction include acyl chlorides, acyl fluorides and acyl bromides. Acyl chlorides are often used. Anhydrides useful in this reaction include the symmetric anhydrides such as acetic anhydride, as well as unsymmetric or ‘mixed’ anhydrides such as formyl pivaloate, or the adduct of a carboxylate anion and a halo-formate such as isobutyl chloroformate. Bases that are useful in the reaction include mildly basic inorganic salts such as sodium or potassium bicarbonate, but typically a soluble base is preferred, such as pyridine, triethylamine, or diisopropyl ethylamine.

Once the protecting group is introduced, the leaving group is replaced with an acetylenic group using the Sonogashira reaction or a similar Pd-catalyzed displacement reaction. These reactions use an acetylenic reagent and a palladium catalyst, usually a soluble Pd species such as tetrakis(triphenylphosphine) palladium or bis(triphenylphosphine) palladium (II) chloride, and typically a copper salt such as copper (I) iodide. The reaction is run in a solvent that is relatively inert under the reaction conditions: ethyl acetate is one suitable solvent for this reaction, but other relatively polar aprotic organic solvents such as acetonitrile, THF, or dioxane may also be used. A base such as a tertiary amine is typically also required, and triethylamine or diisopropyl ethyl amine is often used.

The acetylenic reagent H—C≡C-L can be acetylene itself (L=H), in which case the reaction may be advantageously run at elevated pressure; but it is often more convenient to use trimethylsilyl acetylene or a similar trialkylsilyl acetylene (L=SiR₃) that is a liquid at typical reaction temperatures and can still provide an indole having H at C-2 due to the lability of the silyl group on the acetylene. Where an alkyl or aryl or arylalkyl group is desired at C-2 of the indole target, a terminal acetylene (H—C≡C-L) having that group as L can be employed.

The Sonogashira reaction or other acetylenation reaction can be used to replace PG with an acetylenic substituent, and the ortho-acetylenyl N-protected aniline product can be cyclized into an indole under various conditions. Cyclization of such species to produce an indole is known, and typically uses a copper salt as a catalyst. If a silyl acetylene such as trimethylsilyl acetylene (or another R₃Si group, wherein each R is independently C1-C6 alkyl or phenyl) is used for the acetylenation reaction, the product contains a silyl acetylene and can be cyclized by treatment with a fluoride source in a polar organic solvent such as tetrahydrofuran (THF), dimethoxyethane (DME), or acetonitrile. A tetraalkyl ammonium fluoride salt such as tetrabutylammonium fluoride (TBAF) is often used to effect this cyclization, and acetonitrile is sometimes a preferred solvent for this step. Treatment of the silylacetylene-substituted N-protected aniline with a fluoride source removes the silyl group from the acetylene, and causes cyclization of the ortho-acetylenic aniline to form an indole: the product is thus an indole having no substituent other than H at C2 or C3 when L is a silyl group and fluoride is used to induce cyclization.

Depending upon the choice of the protecting group PG, the cyclization conditions may also remove PG, leaving the newly-formed indole with no substituents at N-1, C-2 or C-3. For example, if PG is an acetyl group and L is a trialkylsilyl group, cyclization to an indole may be accomplished using TBAF in an organic solvent such as acetonitrile or THF. Under these conditions, PG is typically removed during the cyclization reaction; the resulting indole is unsubstituted on the five-membered ring, and can thus be further modified using conventional methods known in the art without requiring a deprotection step.

Once the indole has been constructed, it can be further modified by alkylation at N-1, for example, or by acylation at C-3, or by manipulation of the acyl group at position 5: conditions for many such transformations are known in the art. In one embodiment of the method of the invention, Z in the starting material is an alkoxy, so the 5-position acyl group of the newly formed indole is an ester. In those embodiments, the isolated indole can next be alkylated at N-1 to introduce a group at N-1, using an alkylating agent such as methyl iodide. The ester group can also be hydrolyzed to a carboxylic acid, which can be coupled using known peptide formation reagents to produce an amide that incorporates additional features required for the preparation of a specific target. In a preferred embodiment, such an amide of the 5-position carboxylic acid is formed at N-4 of an N-1-benzyl piperazine such as N-1-(4′-fluorobenzyl)-2S,5R-piperazine, and an N-1 substituent introduced by alkylation as described above is methyl.

In some embodiments, the Z group in Scheme II is an amine so the starting material is an amide of a para-amino-benzoic acid. Z can be a simple amine or an azacyclic group. Typical azacyclic groups within the scope of the invention include pyrrolidines, pyrrolidinones, piperidines, morpholines, piperazines, piperazinones, and the like, each of which is optionally substituted. The azacyclic group in compounds of the invention may be substituted with suitable substitutents such as those typical for an alkyl group, and in some embodiments the azacyclic group is unsubstituted or is substituted with 1-3 groups selected from ═O, methyl, and optionally substituted benzyl.

In some preferred embodiments, the azacyclic group Z in compounds of the invention corresponds to a piperidine or piperazine having a group Ar-L²- at position 4, when defining the point of attachment of the azacyclic ring to the benzoate carbonyl as position 1: Z can therefore provide the piperidine or piperazine ring found in certain of the pharmaceutical compounds referred to above.

In such embodiments, Ar can be an optionally substituted 5-6 membered aromatic ring optionally containing up to two heteroatoms selected from N, O and S; and L² can be a C1-C4 alkylene, or CO or an isostere thereof such as S, SO, SO₂, CH₂, CHOH, CHR or CHOR wherein R is a C1-C4 alkyl group. In some embodiments of Ar-L²-, Ar is optionally substituted phenyl such as, for example, halogen-substituted phenyl including para-fluoro phenyl, and L² is CO or CH₂, often CH₂. In addition to Ar-L²-, the azacyclic group may be substituted with other groups, and a preferred embodiment has Ar-L²- at position 4 of a piperazine along with methyl groups at positions 2 and 5 of the piperazine. One such embodiment is N-1-(4′-fluorobenzyl)-2S,5R-piperazine linked to the carbonyl at position 4 of the piperazine ring. In such embodiments, the azacyclic group often corresponds to the piperidine, piperazine, or pyrrolidine or similar ring of a target pharmaceutical compound, and it may be retained through subsequent steps to produce a compound such as compound no. 33 or compound no. 57 in Table 2 of U.S. Pat. No. 6,867,209, for example. Other suitable azacyclic groups include those required for synthesis of the other compounds in Tables 2 and 3 of U.S. Pat. No. 6,867,209: methods for preparation and incorporation of these azacyclic groups into indoles made using methods described herein are well known.

In other embodiments, Z is a simple amine such as NH₂, NHMe or NMe₂, or an amine group that provides structural features found in an indole target compound, or an alkoxy group that can be used to prepare a pharmaceutical or other target molecule. Often, Z is a methoxy or ethoxy group or another optionally substituted C1-C6 alkoxy group or an optionally substituted phenoxy or aryloxy group that can be readily hydrolyzed to provide a carboxylic acid at C-5 of the indole produced by the methods described herein. These carboxylic acids can be further derivatized using methods known in the art.

Y in the compounds in Scheme II can be H, optionally substituted C1-C6 alkyl, optionally substituted C5-C12 aryl including heteroaryl groups containing 1-3 heteroatoms selected from N, O and S, optionally substituted C6-C13 arylalkyl, an optionally substituted C1-C6 alkylsulfonyl group, an optionally substituted C5-C12 arylsulfonyl group, an optionally substituted C1-C6 alkoxycarbonyl group, or an optionally substituted C1-C6 acyl group. In many cases, the Y group present in a cyclization substrate of formula (II) will also be present at the indole nitrogen of the cyclized product of formula (III). Where Y is an acyl group in a compound of formula (II), however, the product of formula (III) will typically contain H rather than Y at the indole nitrogen if the cyclization is accomplished using fluoride to cyclize a compound of formula (II) wherein L is a silyl group R₃Si.

In certain embodiments of the reaction in Scheme II, X represents at least one substituent in addition to LG or the acetylenic group, the amine and the carboxylate derivative —C(O)Z. Typically, X represents one substituent, and in many embodiments of the invention, X is a single substituent that is ortho to the —C(O)Z group. In some embodiments, X is ortho to —C(O)Z in a para-aminobenzoate derivative of formula (I); and LG is an iodide group (—I) that is introduced by iodination:

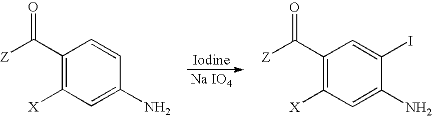

This places the —I group para to X, and provides a para-amino benzoate derivative that is a suitable starting material for use in the methods of the invention once it is protected. In some embodiments of this type, X is C1 or CF₃, or methyl or C1-C4 alkoxy: X=Cl, Me or OMe is sometimes selected for use in these methods, because such intermediates are ideal precursors for certain pharmaceuticals; X=Cl is sometimes selected.

Several variations of this method have also been developed. For example, it has been found that complete protection of an aniline to be cyclized is not required in order to get the cyclization reaction to work well. While not being bound by any theory or mechanism, it appears that the fluoride-induced cyclization conditions can catalyze acyl transfer between the amine groups of molecules in the reaction mixture. As a result, if at least a catalytic fraction of the aniline starting material is acylated with a C1-C6 acyl group, the reaction proceeds nearly as well as if the starting material were fully acyl-protected. Furthermore, if the starting material is over-acylated (e.g., in a compound of formula (II) where Y and PG are both C1-C6 acyl groups), the reaction also proceeds well—presumably because the fluoride-containing reaction conditions facilitate acyl transfer resulting in deacylation of a diacyl aniline to an extent sufficient to permit cyclization of a mono-acyl species. As a result, it is possible to produce indoles according to Scheme III using a catalytic amount of an acyl or diacyl aniline derivative admixed with a substrate of formula IV even if the substrate of formula (IV) is not protected (i.e., if R″ represents H). Where R′ is a silyl group R₃Si in formulas (IV) and (V), the cyclization as illustrated in Scheme III can be accomplished with a fluoride source in an organic solvent to produce an indole of formula (VI) in which R′ on the indole is H.

Thus in one aspect, the invention provides methods to synthesize indoles of formula (VI) from unprotected ortho-acetylenyl anilines of formula (IV) by using a catalytic amount of an acyl aniline of formula (V):

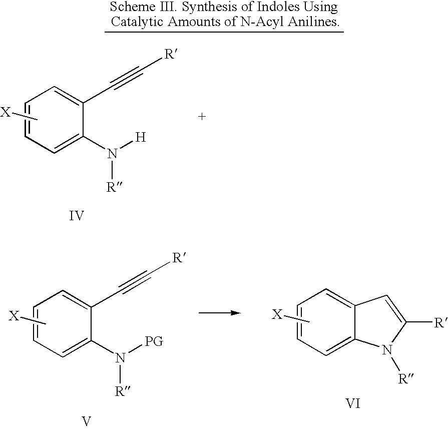

wherein:
X represents 0-3 substituents suitable for an aryl group;
PG represents an optionally substituted C1-C6 acyl group;
each R′ independently represents H, optionally substituted C1-C6 alkyl, optionally substituted C5-C12 aryl including heteroaryl groups containing 1-3 heteroatoms selected from N, O and S, optionally substituted C6-C13 arylalkyl, or R₃Si wherein each R is independently C1-C6 alkyl or phenyl;
each R″ independently represents H, optionally substituted C1-C6 alkyl, optionally substituted C5-C12 aryl including heteroaryl groups containing 1-3 heteroatoms selected from N, O and S, optionally substituted C6-C13 arylalkyl, an optionally substituted C1-C6 alkylsulfonyl group, an optionally substituted C5-C12 arylsulfonyl group, or an optionally substituted C1-C6 alkoxycarbonyl group.

The reaction shown in Scheme III can, of course, be applied to preparing indoles of formula (III) if the compound of formula (IV) is a para-aminobenzoate derivative. Thus X in certain embodiments of the method depicted in Scheme III represents an alkoxycarbonyl or aminocarbonyl having the same structures as an ester or amide portion of the para-aminobenzoate derivatives described for the compounds of Scheme II. In addition, since X can represent multiple substituents, in some embodiments of this method the compounds are of the following formula, which represents a preferred embodiment of this method:

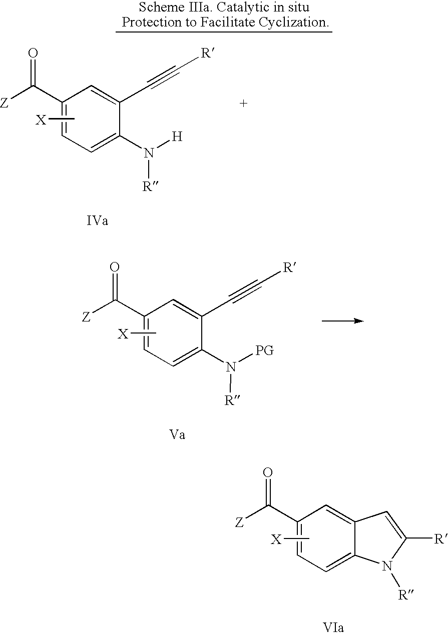

wherein Z and X are as defined for compounds in Scheme II, and R′ and R″ are as defined for compounds in Scheme III. Often in these embodiments, Z is a C1-C6 alkoxy group.

Preferably, R′ is the same in formulas (IV) and (V), and R″ is the same in formulas (IV) and (V); however, R″ and R′ in the product of formula (VI) will be determined by the cyclization conditions and can differ from those features in the starting material.

In Schemes III and IIIa, typically the R′ group in the compound of formula (IV) and (V) is a trialkylsilyl such as trimethylsilyl, in which case R′ in the product of formula VI is typically H. R″ in the compound of formula IV is typically H, so the reaction product VI will also have R″=H. In one embodiment, R″ is H in the compound of formula IV and R″ is either H or the same as PG in the compound of formula V; the product in that embodiment is a compound of formula VI having R″=H.

Thus in another aspect, the invention provides methods for cyclizing a compound of formula (IV) or (IVa) to produce an indole of formula (VI) or (VIa), using a catalytic amount of a compound of formula (V) or (Va). Typically, the compound of formula (V) or (Va) is a protected form of the compound of formula (IV) or (IVa): thus a sample of a compound of formula (IV) or (IVa) could be mono-acylated or di-acylated using standard methods for aniline protection, and this material would serve as a catalyst during the cyclization of the compound of formula (IV) or (IVa).

The amount of the protected compound of formula (V) or (Va) used may vary from about 5% or about 10% to about 20% or about 30%, and typically does not exceed about 50%. The compound of formula (V) has at least one protecting group PG, which is an optionally substituted C1-C6 acyl group. R″ in Scheme III can be H, or an alkyl or aryl group or an alkyl- or arylsulfonyl group that will remain in the product; or it can be an acyl group similar to or the same as PG, in which case the product of formula (VI) will typically have R″=H, i.e., where both PG and R″ are acyl groups, typically both will be removed under the cyclization conditions. Preferably, R″ is H and PG is an acetyl or formyl group; or R″ in the compound of formula (IV) or (IVa) is H, and R″ in the compound of formula (V) is the same as PG and is acetyl or formyl. In each of these cases, if R′ is a silyl group R₃Si, and the cyclization is accomplished using fluoride, the product of formula (VI) of (Via) will contain R″=H.

The following examples are offered to illustrate but not to limit the invention.

### EXAMPLE 1

### Synthesis of 2-Chloro-4-acetamido-5-iodobenzoic Acid Methyl Ester

The methyl ester of 2-chloro-4-amino-5-iodobenzoic acid (ca. 362 kg) was mixed with tetrahydrofuran (THF: 826 kg) and stirred. Pyridine (138 kg) and more THF (92 kg) were added, and the mixture was cooled to 10-20° C. Acetyl chloride (119 kg) was added at a rate that permitted the temperature to be maintained below 25° C.; THF (92 kg) was used to rinse the acetyl chloride into the reaction vessel. The reaction mixture was stirred for 4 hours or until at least 97% consumption of starting material was achieved as judged by HPLC analysis.

The mixture was cooled to 10-15° C., and water (2520 L) was added in 3 batches, while the temperature was maintained at 10-25° C. The solids were then collected by processing the mixture in five batches with a centrifuge: each batch was washed with water (91 L), water (91 L), and methanol (91 L). After this, each batch was suspended in methanol (2500 L) and refluxed for one hour, and was then cooled to 0-5° C. Product was collected by centrifugation and dried at 35-45° C. If analysis of a batch shows a significant amount of the regioisomeric iodide or the diiodide carried through from the iodination step (e.g., if the product constitutes less than about 93% of the material detected by HPLC), the solids may be slurried with cold methanol and re-centrifuged to remove much of those impurities.

Repeating this reaction at about this scale several times provided a reproducible yield of 78-81%, and the product was an off-white to light brown or light pink solid that was 97-99% homogenous by HPLC.

### EXAMPLE 2

### Preparation of 2-Chloro-4-acetamido-5-(2-trimethylsilylethynyl)-benzoic Acid Methyl Ester

2-Chloro-4-acetamido-5-iodobenzoic acid methyl ester (ca. 370 kg), bis(triphenylphosphine)Pd(II) chloride (3.84 kg), and copper (I) iodide (1.06 kg) were combined in a reaction vessel, and ethyl acetate (2048 kg) was added. The mixture was held at 28-32° C. while trimethylsilyl acetylene (122 kg) was added; this was rinsed in with 100 kg ethyl acetate. Triethylamine (126 kg) was then added, with the temperature kept below 40° C., and the mixture was stirred at 38-42° C. for 4 hours or until less than 1% starting material remained by HPLC.

The mixture was then cooled to 15-25° C., and water (1100 L) was added; after stirring for 30 minutes, the aqueous layer was removed and discarded, and the aqueous wash was repeated with another 1100 L portion of water.

It appears that treatment of this solution with Cuno carbon may be useful to reduce the amount of palladium residue remaining in the product and to improve the purity and color at later stages in the reaction sequence. In addition, an aqueous EDTA wash can be used at this stage to remove most of the copper salts present.

The organic layer was then vacuum distilled without heating above 35° C. until the volume reached about 720 L. Heptane (937 kg) was added, and the vacuum distillation was repeated until the volume reached about 1400 L, removing most of the ethyl acetate present and crystallizing out the product. A second addition of heptanes and vacuum distillation may be added at this stage to further reduce the amount of ethyl acetate present, which may improve the yield without adversely affecting purity.

This mixture was then cooled to 0-5° C., and processed in five batches for product isolation. Each batch was spun down in a centrifuge, and was then rinsed with 186 L heptane. The wet cakes were combined for a yield of 84-87% averaged over several repetitions at about this scale (260-330 kg). The product was an off-white to brown powder that was 97-99+% pure as judged by HPLC.

### EXAMPLE 3

### Preparation of 6-Chloro-5-methoxycarbonyl Indole

A solution of tetrabutylammonium fluoride (TBAF) in acetonitrile was prepared by dissolving 372 kg of TBAF trihydrate in acetonitrile (720 kg) with stirring for at least one hour.

2-Chloro-4-acetamido-5-(2-trimethylsilylethynyl)-benzoic acid methyl ester (ca. 300 kg) was dissolved in acetonitrile (1168 kg) at 20-30° C. The TBAF/acetonitrile solution was added to this solution with stirring, while the temperature was maintained at less than 35° C. The TBAF solution was rinsed in with another portion (49 kg) of acetonitrile.

This mixture was heated to 78-80° C. for 7 hours or until less than 1% starting material remained as judged by HPLC, and the product accounted for at least 93% of the material by HPLC. The mixture was then vacuum distilled at a temperature not exceeding 35° C. to a volume of about 1200 L. It was then split into two batches, each of which was combined with about 1900 L water at 15-25° C. for one hour to precipitate the product. About half of each batch was spun down in a peeler centrifuge and rinsed with water (198 L); then the other half of each batch was processed similarly. When all of both batches had been processed, the collected off-white to pale brown solids were dried at 35-40° C. to provide a 91-92% yield of material that was 95-97% pure as judged by HPLC.

Laboratory work suggests that this yield may be improved by reducing the volume of acetonitrile present, as by vacuum distillation, prior to the aqueous quench, and by increasing the volume of water used for the quench. This has not been tested on large scale, however.

The above examples are simplified descriptions of representative scale-up and pilot plant runs for each of these specific reactions. More detailed descriptions of these examples, including comparison of multiple batches up to pilot plant scale, are presented in the following examples.

### EXAMPLE 4

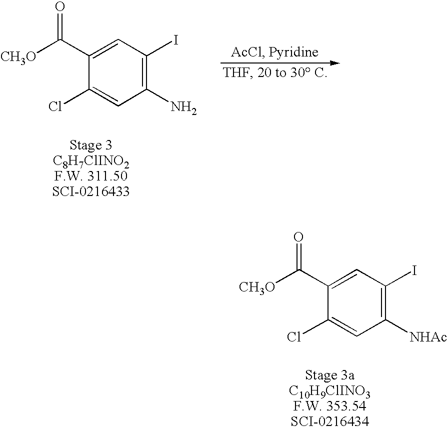

The Stage 3a process has undergone a number of changes since Campaign S2. These are summarised briefly below:

For Campaigns S2 and S3, an optional methanol re-slurry was included in the Stage 3a process, dependent on the HPLC purity of the crude wet-cake. If the wet-cake purity was below 93 area % then the re-slurry was performed. This was the case for both S2 and S3 and it was decided that the re-slurry should be made mandatory for S4 for the sake of process consistency. In the event, the crude Stage 3a product was much purer than in previous campaigns (97.6 area %) and the re-slurry upgraded it to 99.9 area %. Mass balance studies indicated that the re-slurry resulted in a yield loss of around 5%.

For Campaign S5, the Stage 3a process was transferred to the RPS Dudley One Semi-works facility. The batch size (Stage 3 input) was increased by a factor of 2.3 from 127 to 292 kg. Isolation of the product was carried out using a peeler centrifuge in place of the ceramic vat-filter employed in the Pilot Campaigns.

As the S5 product was to be isolated on a centrifuge, it was anticipated that the crude material would have a greater purity than that seen in earlier campaigns and that the methanol re-slurry would therefore not be required. This proved to be the case and all the crude wet-cake from both batches was found to have purity in excess of 93 area %. In fact, eight out of ten of the centrifuge baskets had purities greater than 98%. Centrifugation was clearly more efficient at liquor removal than vat-filtration.

The batch size (Stage 3a input) for Campaign S6 was increased by a factor of 1.31 from 292 to 382 kg. The only other change was the incorporation of additional cold methanol centrifuge washes for one of the batches, which had an abnormally high regioisomer content in the Stage 3 input. This strategy proved successful as the regioisomer content was reduced from as high as 12.9 area % in the Stage 3 input to less than 1 area % in the Stage 3a product.

Comparison of Yield and Purity for Campaigns S3 to S6.

Table One gives a comparison of yield and purity data for Campaigns S3 to S6.

TABLE ONE
Stage 3a Yield and Purity Data for Campaigns S3 to S6

| | | | | | Camp'n | Stage 3a |
|---|---|---|---|---|---|---|
| | | | | Camp'n | Assay- | HPLC |
| | | | Stage 3a | Physical | based | Purity |
| | Stage 3a Batch | Stage 3 Input | Output | Yield | Yield | (Area |
| Campaign | Number | (kg) | (kg) | (%) | (%) | %) |
| S3 | 80009407A, B | 122.7 | 102.5 | 73.6 | Data not | 94.4 |
| | | | | | available | |
| S4 | 80010873A, B | 127.0 | 114.7 | 79.6 | 83.8 | 99.9 |
| S5 | 80012347A, B | 291.9 | 274.1 | 80.8 | 82.4 | 99.1 |
| | 80012348A, B, C | 291.9 | 261.2 | | | 98.7 |
| S6 | 80012940A, B, C | 374.0 | 343.6 | 78.0 | 81.6 | 96.6 |
| | 80012941A, B, C | 391.0 | 333.8 | | | 99.0 |

The assay-based yields have remained fairly constant across the four campaigns S3 to S6. Laboratory experiments indicate that yields in excess of 90% should be achieved in the absence of the methanol re-slurry. Campaigns S3 and S4 both incorporated the re-slurry, which was undoubtedly the main reason for the low yields observed.

Surprisingly, no improvement in yield was seen in S5, which did not incorporate the re-slurry. Due to the relatively high minimum stir volume in the reaction vessel V3166, it proved necessary to charge additional THF (29% of original charge) in order to achieve efficient agitation. Although the water quench charge was increased by the same proportion, this change resulted in the Stage 3a product being isolated from a significantly increased solvent volume. This is probably the main reason for the yield short-fall in S5. However, analysis of the mother liquors did not indicate the presence of significant amounts of product. It has since been shown that the assay method underestimates the amount of Stage 3a present when applied to solutions. In future, liquor samples should be concentrated to dryness before attempting to perform an assay.

The S6 yield was even more disappointing than that obtained in S5. There are two principle reasons for this:

The Stage 3 input for the second batch had an abnormally high level of the regioisomeric impurity (up to 12.9 area %). In order to remove this, additional cold methanol washes were applied during centrifugation of the Stage 3a product. These proved highly effective in removing the impurity, but undoubtedly had a detrimental effect on yield.

For batch 80012941, additional THF had to be charged in order to mobilise solids remaining in vessel V3166 after transfer of the main batch to V1001. The solids were successfully mobilised, but it was necessary to charge additional water as an anti-solvent to compensate for the extra THF. In spite of the additional water charge, it is likely that this deviation will have resulted in some loss of product to the liquors.

To prevent a repetition of the losses observed in S5 and S6, the following measures are proposed or have already been implemented:

Particular attention should be paid to isolation of the Stage 3 product on the centrifuge. Efficient de-liquoring at this point is essential to removing the unwanted regioisomer and thereby avoiding the need for additional methanol washes at Stage 3a.

Larger agitator blades have been fitted to V3166. The resulting improved agitation should avoid the need to charge additional THF to the reaction mixture as in S5.

The reaction mixture should be quenched with water in V3166 before transfer to V1001. This should avoid the problems associated with solids settling out in V3166 during the transfer. Once quenched, the mixture is much more mobile and should be transferred more readily,

In addition to the above changes, an increased reaction concentration has now been demonstrated at Pilot scale. This will result in a significant improvement in volumetric efficiency and allow a larger batch size to be accommodated.

Process Outline

Below is the process outline of the Stage 3a process employed in Campaign S6. Process outlines for Campaigns S3, S4 and S5 are contained in the relevant campaign reports.

| Step | Description |
|---|---|
| 1. | Charge N-methyl indole acid Stage 3 (362 Kg) to vessel V3166 via the solids |
| | charging chute. |
| 2. | Charge tetrahydrofuran (56 Kg) to vessel V3166 via solids charge chute as a |
| | rinse. |
| 3. | Charge tetrahydrofuran (770 Kg) to vessel V3166 via drums. |
| 4. | Start V3166 agitator. |
| 5. | Charge pyridine (138 Kg) to vessel V3166 via drums. |
| 6. | Charge tetrahydrofuran (92 Kg) to vessel V3166, via drums, as a line-wash. |
| 7. | Cool contents of vessel V3166 to 10-20° C. |
| 8. | Charge acetyl chloride (119 Kg) to dispenser R3173. |
| 9. | Transfer contents of dispenser R3173 to vessel V3166 at a rate that maintains the |
| | batch temperature in the range 10 to 25° C. |
| 10. | Charge tetrahydrofuran (92 Kg) to vessel V3166, via dispenser R3173, as a line |
| | wash. |
| 11. | Stir contents of vessel V3166 at 20 to 30° C. for NLT 4 hours. |
| 12. | Sample contents of vessel V3166 for completion (DI/SD469/306). |
| | Limit: NMT 3% Stage 3 by HPLC area %. |
| | If complete, continue processing at Step 21. |
| | If incomplete, continue processing at Step 13. |
| 13. | Stir contents of vessel V3166 at 20 to 30° C. for NLT 2 hours. |
| 14. | Sample contents of vessel V3166 for completion (DI/SD469/306). |
| | Limit: NMT 3% Stage 3 by HPLC area %. |
| | If complete, continue processing at Step 21. |
| | If incomplete, continue processing at Step 15. |
| 15. | Charge pyridine (quantity specified by chemist) to vessel V3166 via dispenser |
| | R3173 maintaining the batch temperature in the range 20 to 30° C. |
| 16. | Charge tetrahydrofuran (45 Kg) to vessel V3166 via dispenser R3173. |
| 17. | Charge acetyl chloride (quantity specified by chemist) to vessel V3166 via |
| | dispenser R3173 maintaining the batch temperature in the range 20 to 30° C. |
| 18. | Charge tetrahydrofuran (45 Kg) to vessel V3166 via dispenser R3173. |
| 19. | Stir contents of vessel V3166 at 20 to 30° C. for NLT 2 hours. |
| 20. | Sample contents of vessel V3166 for completion (DI/SD469/306). |
| | Limit: NMT 3% Stage 3 by HPLC area %. |
| | If complete, continue processing at Step 21. |
| | If incomplete, consult chemist. |
| 21. | Transfer contents of vessel V3166 to vessel V1001. |
| 22. | Charge tetrahydrofuran (92 Kg) to vessel V3166 via drums. |
| 23. | Transfer contents of vessel V3166 to vessel V1001 as a line rinse. |
| 24. | Cool contents of vessel V1001 to 10 to 15° C. |
| 25. | During the cool back, charge water (631 L) to vessel V1 via bulk supply. |
| 26. | Charge contents of V1 to vessel V1001 over NET 1 hour, maintaining the batch |
| | temperature in the range 10 to 25° C. |
| 27. | Charge water (1261 L) to vessel V3166 via bulk supply and stir for NLT 30 |
| | minutes. |
| 28. | Transfer contents of V3166 to vessel V1001 maintaining the batch temperature in |
| | the range 10 to 25° C. |
| 29. | Charge water (631 L) to vessel V3166 via bulk supply and stir for NLT 30 |
| | minutes. |
| 30. | Transfer contents of V3166 to vessel V1001 maintaining the batch temperature in |
| | the range 10 to 25° C. |
| 31. | Agitate contents of vessel V1001 at 20-25° C. for NLT 2 hours. |
| 32. | Transfer approximately one fifth of the contents of vessel V1001 to centrifuge |
| | D3353. Collect liquors in receiver R3350. |
| 33. | Charge water (91 L) to header A3160 via drums. |
| 34. | Transfer contents of header A3160 to centrifuge D3353 as a cake wash, |
| | collecting liquors in receiver R3350. |
| 35. | Charge water (91 L) to header A3160 via drums. |
| 36. | Charge methanol (91 L) to header A3160 via bulk supply. Circulate to mix. |
| 37. | Transfer contents of header A3160 to centrifuge D3353 as a cake wash, |
| | collecting liquors in receiver R3350. |
| 38. | Drum up contents of receiver R3350 for disposal. |
| 39. | Discharge solids from centrifuge D3353 to kegs. |
| 40. | Repeat steps 32 to 39 three more times. |
| 41. | Transfer remaining contents of vessel V1001 to centrifuge D3353. Collect liquors |
| | in receiver R3350. |
| 42. | Charge water (91 L) to vessel V1001 via drums. |
| 43. | Transfer contents of vessel V1001 to centrifuge D3353 as a cake wash and line |
| | rinse, collecting liquors in receiver R3350. |
| 44. | Charge water (91 L) to header A3160 via drums. |
| 45. | Charge methanol (91 L) to header A3160 via bulk supply. Circulate to mix. |
| 46. | Transfer contents of header A3160 to centrifuge D3353 as a cake wash, |
| | collecting liquors in receiver R3350. |
| 47. | Drum up contents of receiver R3350 for disposal. |
| 48. | Discharge solids from centrifuge D3353 to kegs. |
| 49. | Sample wet cake for purity. (DI/SD469/307). |
| | Limit: NLT 93 area % Stage 3a |
| | If the analysis passes, continue at Step 83. |
| | If the analysis fails, and this is the first batch of the campaign, continue at step |
| | 50. |
| | If the analysis fails, and this is the second batch of the campaign, continue at step |
| | 50, but omit steps 74-82. |
| 50. | Charge wet cake to vessel V3166 via the charge chute. |
| 51. | Charge methanol (204 L, 1 full drum) to vessel V3166 via solids charge chute as |
| | a line rinse. |
| 52. | Charge bulk methanol (1453 L) to vessel V3166. |
| 53. | Start agitator and stir contents of V3166 for at least 15 minutes. |
| 54. | Transfer contents of V3166 to V1001. |
| 55. | Start V1001 agitator. |
| 56. | Charge bulk methanol (854 L) to vessel V3166, and stir for at least 15 |
| | minutes. |
| 57. | Transfer contents of V3166 to V1001 as vessel rinse. |
| 58. | Heat contents of vessel V1001 to reflux (65-67° C.). |
| 59. | Stir contents of vessel V1001 at reflux for NLT 1 hour. |
| 60. | Cool contents of vessel V1001 to 0 to 5° C. over about 2 hours. |
| 61. | Stir contents of vessel V1001 at 0 to 5° C. for NLT 1 hour. |
| 62. | During coolback charge bulk methanol (700 L) to header A3160 |
| 63. | Cool contents of header A3160 to 0 to 5° C. |
| 64. | Transfer approximately one fifth of the contents of vessel V1001 to centrifuge |
| | D3353. Collect liquors in receiver R3350. |
| 65. | Transfer methanol (approximately 130 L) from header A3160 to centrifuge |
| | D3353 as a cake wash, collecting the liquors in receiver R3350. |
| 66. | Spin dry and discharge solids from centrifuge D3353 to kegs. |
| 67. | Repeat steps 64 to 66 three more times |
| 68. | Transfer remaining contents of vessel V1001 to centrifuge D3353. Collect liquors |
| | in receiver R3350. |
| 69. | Charge methanol (130 L) to vessel V1001 via bulk supply. |
| 70. | Cool contents of vessel V1001 to 0 to 5° C. |
| 71. | Transfer the contents of vessel V1001 to centrifuge D3353 as a cake wash and |
| | line rinse, collecting the liquors in receiver R3350. |
| 72. | Spin dry and discharge solids from centrifuge D3353 to kegs. |
| 73. | Transfer remaining contents of header A3160 directly to receiver R3350 and |
| | drum up R3350 contents for disposal. |
| 74. | V3166 Boil-out Between Batches |
| | Charge tetrahydrofuran (573 L —3 full drums) to vessel V3166 via drums. |
| 75. | Start agitator and set contents of vessel V3166 to reflux. |
| 76. | Allow contents of vessel V3166 to reflux for NLT 1 hour. |
| 77. | Cool contents of vessel V3166 to 25 to 30° C. |
| 78. | Transfer contents of vessel V3166 to vessel V1001. |
| 79. | Bake vessel V3166 dry under vacuum |
| 80. | Transfer contents of V1001 to receiver R3350. |
| 81. | Cool contents of vessel V1001 to 15 to 25° C. |
| 82. | Drum up contents of receiver R3350 for disposal. |
| 83. | Charge wet-cake to dryer K3045, keeping baskets separate. |
| 84. | Dry wet-cake in dryer K3045 at 35 to 45° C. until an LOD of NMT 0.5% is |
| | achieved. |
| 85. | Discharge product from dryer K3045 to drums. |

Expected dry weight = 333 Kg (81%)

Equipment Train

For Campaigns S3 and S4, Stage 3a manufacture, aqueous work-up and crystallisation were performed in PV351, a 300-gallon glass-lined vessel. The product was isolated in PD351 and PD404, ceramic vat-filters associated with PV351 and dried in PK602, a double cabinet air tray-dryer.

Campaigns S5 and S6 were carried out in the RPS Dudley One Semi-works facility. The reaction chemistry took place in V3166, a 1000-gallon hastelloy reactor. The aqueous quench/crystallisation was performed in V1001, a 1000-gallon glass-lined vessel, and the product was isolated on D3353, a hastelloy peeler centrifuge. Drying took place in K3045, a stainless steel vacuum tray dryer.

Quality-Critical Parameters

No laboratory work to identify quality-critical parameters has been performed for Stage 3a. The product manufactured in Campaigns S3 to S6 has consistently satisfied specification and no quality-critical parameters have been identified from the processing records.

The quality of the Stage 3a manufactured is largely determined by the quality of the Stage 3 input. In Campaign S3 and one batch of S6, relatively high levels of diiodo-Stage 3 were present in the Stage 3 input. This impurity was partially removed during the Stage 3a process, but not completely. This is reflected in the low purities for the relevant batches shown in Table One. Clearly, good control over formation of the diiodo impurity at Stage 3 is essential to generating high purity Stage 3a.

The other main impurity observed in Stage 3a is the Stage 3a regioisomer. This is the product of acetylation of the corresponding Stage 3 impurity. Efficient centrifugation of the Stage 3 product is the main requirement for controlling the level of this impurity.

As indicated earlier, the Stage 3a process includes provision for a methanol re-slurry if the wet-cake purity is below 93 area %. In reality, centrifugation results in high purity product and the re-slurry was employed in neither S5 nor S6.

Parameters Affecting Yield

In Campaigns S5 and S6, it proved necessary to charge additional THF to the process in order to achieve satisfactory slurry mobility. Although additional water (anti-solvent) was charged pro-rata to compensate for this, it seems likely that the additional solvent resulted in some yield losses. As described above, improved agitation and direct quenching of the batch in V3166 should remove the need for extra THF in future campaigns.

The Stage 3 input for S6 batch 80012941A,B,C contained a high level of the Stage 3 regioisomer. This was acetylated to give the Stage 3a regioisomer and extra methanol centrifuge washes were applied to remove it from the product. These undoubtedly resulted in some yield loss. By ensuring efficient regioisomer removal at Stage 3, it should be possible to prevent such losses in future campaigns.

Conversion of Stage 3 to Stage 3a is monitored by HPLC analysis of the reaction mixture. Table Two gives a comparison of final in-process levels of Stage 3 and the Stage 3 content of the isolated Stage 3a product. The current in-process limit for unreacted Stage 3 is 3 area % and this has remained unchanged since the process was introduced to the Dudley site in 2001.

TABLE TWO
Comparison of In-process and Intermediate Stage 3 levels for Stage 3a
Campaigns S3 to S6

| | | Final In-process | Stage 3 in Isolated |
|---|---|---|---|
| | | Stage 3 | Stage 3a |
| Campaign | Batch | (HPLC Area %) | (HPLC Area %) |
| S3 | 80009407A, B | 1.0 | A - ND |
| | | | B - ND |
| S4 | 80010873A, B | ND | A - ND |
| | | | B - ND |
| S5 | 80012347A, B | ND | A - ND |
| | | | B - ND |
| | 80012348A, B, C | ND | A - ND |
| | | | B - ND |
| | | | C - 0.9 |
| S6 | 80012940A, B, C | 2.5 | A - ND |
| | | | B - ND |
| | | | C - 0.9 |
| | 80012941A, B, C | 0.3 | A - ND |
| | | | B - ND |
| | | | C - 0.2 |

The data in Table Two indicate that in-process levels of unreacted Stage 3 up to 2.5 area % are well tolerated by the process. The S6 batch 80012940 gave a final completion result of 2.5 area % Stage 3, but the batch still had less than 1.0 area % of Stage 3 in the isolated product. It would therefore seem that the unreacted starting material is efficiently removed during isolation. From an economic viewpoint, this represents significant lost yield. It is therefore recommended that the in-process limit be lowered to 1 area %. There should be no difficulty in achieving this level. In fact, the lower limit has already been employed successfully in the recent Pilot Trial.

TABLE THREE
Comparison of Stage 3a In-process and Intermediate HPLC
Purities for Campaigns S3 to S6

| | | In-Process | Intermediate | Methanol |
|---|---|---|---|---|
| | | HPLC Purity | HPLC Purity | Re-slurry |
| Campaign | Batch | (Area %) | (Area %) | Employed? |
| S3 | 80009407A, B | 89.4 | 94.4 | Yes |
| S4 | 80010873A, B | 97.6 | 99.9 | Yes^{a} |
| S5 | 80012347A, B | 99.0^{b} | 99.1^{c} | No |
| | 80012348A, B, C | 97.5^{b} | 98.7^{c} | No |
| S6 | 80012940A, B, C | 96.8^{b} | 96.6^{c} | No |
| | 80012941A, B, C | 99.3^{b} | 99.0^{c} | No |

^{a}Re-slurry performed, even though batch met in-process limit, for consistency with previous batches
^{b}Values shown are simple averages for all centrifuge basket-loads
^{c}Values shown are simple averages for all part-batches (A, B etc.) corresponding to oven charges

All batches from Campaign S4 onwards have readily achieved the in-process limit and have therefore not required re-slurry. However, a re-slurry was employed for the S4 batch for the sake of consistency with earlier batches. The Stage 3a product isolated on the centrifuge in S5 and S6 had generally high purity (>98 area %). Batch 80012940 had atypically low purity (96.6 area %) on account of a high level of the diiodo impurity carried over from Stage 3. The high purity of batch 80012941 was ensured by employing additional methanol centrifuge washes to remove the regioisomeric impurity, which was present at high levels due to inefficient centrifugation of the Stage 3 input.

With efficient centrifugation and good control over formation of the diiodo impurity at Stage 3, it should be possible to generate Stage 3a product with greater than 98 area % purity. Up to and including Campaign S6, the Stage 3a purity specification was set very low at ‘NLT 85 area %’. It has since been tightened to ‘NLT 94 area %’. The methanol re-slurry is known to be effective in upgrading the Stage 3a purity by removing the regioisomeric impurity and, to a lesser extent, the diiodo impurity. It would therefore be prudent to retain the in-process analysis, raising the limit to ‘NLT 94 area %’. This would allow material which failed to meet the purity specification to be upgraded before drying.

TABLE FOUR
Comparison of Stage 3a In-process and Intermediate LOD
Values for Campaigns S3 to S6

| | | Final In-process | Intermediate LOD |
|---|---|---|---|
| Campaign | Batch | LOD (% w/w) | (% w/w) |
| S3 | 80009407A, B | 0.2^{a} | A - ND |
| | | | B - ND |
| S4 | 80010873A, B | 0.1^{a} | A - 0.1 |
| | | | B - 0.1 |
| S5 | 80012347A | 0.05 | ND |
| | 80012347B | 0.13 | 0.02 |
| | 80012348A | 0.14 | ND |
| | 80012348B | 0.12 | 0.05 |
| | 80012348C | 0.06 | 0.36 |
| S6 | 80012940A | ND | 0.05 |
| | 80012940B | ND | 0.04 |
| | 80012940C | 0.1 | 0.04 |
| | 80012941A | ND | 0.04 |
| | 80012941B | 0.1 | 0.05 |
| | 80012941A | 0.1 | 0.04 |

^{a}In-process sample was composite of parts A and B

Table Four shows good agreement between in-process and intermediate LOD values for Stage 3a. Based on this data, the current in-process limit is judged adequate to ensure that the intermediate specification (NMT 0.5% w/w) is achieved.

Stage 3a HPLC Profile

Table Five shows the Stage 3a HPLC profiles for Campaigns S3 to S6. The overall purity has generally been greater than 94 area %. In fact, when good control over the regioisomer and diiodo impurities is achieved, purity values in excess of 98 area % have been consistently observed. The overall purity of the Stage 3a product is less important than the levels of the individual impurities. For example, it is known that the diiodo impurity up to a level of 5.8 area % is removed at Stage 4. In contrast, there is no data to support onward processing of such high levels of the Stage 3a regioisomer.

Stage 3a Regioisomer

This impurity is formed by acetylation of the 3-iodo regioisomer formed as a major side-product at Stage 3. The level of the impurity is therefore determined by the amount of the 3-iodo compound present in the Stage 3 input. This, in turn, is determined by the efficiency of liquor removal and washing on the centrifuge. Good centrifuge technique at Stage 3 is therefore essential to controlling this impurity. If the impurity is detected at high levels in the Stage 3 product, it can be removed by means of an isopropyl alcohol re-slurry. It can also be removed at Stage 3a, by re-slurrying the wet-cake in methanol.

Diiodo Impurity

This impurity is generated at Stage 3 as a result of over-iodination. As yet, there is no proven method for removing it, although the Stage 3a methanol re-slurry is partially effective. Levels up to 5.8 area % have been tolerated by the Stage 4 process (Campaign S3). It is thought that the impurity is converted to the bis(TMS acetylene) compound under the Stage 4 conditions and that this is removed during the crystallisation. The current specification is ‘Report value’.

RRTs 0.83 and 1.09

These impurities have only been detected in two part-batches. Their identity is not known.’

TABLE FIVE
Stage 3a HPLC Profiles for Campaigns S3 to S6

| | | Component (Area %) | | | | | |
|---|---|---|---|---|---|---|---|
| | | | | Stage 3a | | | |
| | | | RRT | Regio- | | | Diiodo |
| Campaign | Batch | Stage 3 | 0.83 | isomer | RRT 1.09 | Stage 3a | Impurity |
| S3 | 80009407A | ND | ND | ND | ND | 94.2 | 5.8 |
| | 80009407B | ND | ND | ND | ND | 94.5 | 5.5 |
| S4 | 80010873A | ND | ND | ND | ND | 100.0 | ND |
| | 80010873B | ND | 0.21 | <0.1 | ND | 99.7 | ND |
| S5 | 80012347A | ND | ND | ND | ND | 99.13 | 0.87 |
| | 80012347B | ND | ND | ND | ND | 99.12 | 0.88 |
| | 80012348A | ND | ND | ND | ND | 99.25 | 0.75 |
| | 80012348B | ND | ND | ND | ND | 99.24 | 0.76 |
| | 80012348C | 0.9 | ND | 0.12 | ND | 97.9 | 1.1 |
| S6 | 80012940A | ND | ND | <0.1 | ND | 97.6 | 2.4 |
| | 80012940B | ND | ND | <0.1 | ND | 97.0 | 2.9 |
| | 80012940C | 0.9 | ND | <0.1 | 0.58 | 95.3 | 3.2 |
| | 80012941A | ND | ND | 0.4 | ND | 99.6 | ND |
| | 80012941B | ND | ND | 0.5 | ND | 99.2 | 0.7 |
| | 80012941C | ND | ND | 0.8 | ND | 98.2 | 1.0 |

### EXAMPLE 5

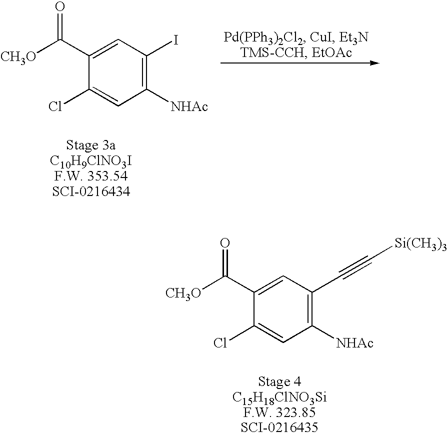

For Campaign S3, the reaction stir-out period was extended from 4 to 8 hours in order to ensure complete consumption of the 3,5-diiodo impurity, which was present at an atypically high level (5.7 area %) in the Stage 3a product. The change was successful, in that the isolated Stage 4 product contained no detectable diiodo impurity. The change was purely temporary, due to the abnormal nature of the Stage 3a input. Subsequent campaigns reverted to the 4-hour reaction period.

For Campaign S4, the product isolation procedure was modified to eliminate problems observed in Campaigns S2 and S3. The first vacuum distillation was terminated at a higher residual volume to prevent ‘caking’ of the product on the vessel walls as seen in S2. Heptane was then charged and a second vacuum distillation was performed to remove residual ethyl acetate. By ensuring adequate removal of ethyl acetate, it was anticipated that the yield losses seen in S3 would be avoided. The change proved highly successful. An excellent physical yield (96.0%) was achieved in S4 and very little product adhered to the vessel walls during the isolation procedure. Satisfactory product purity was maintained.

Stage 4 manufacture for Campaign S5 was scaled up by a factor of 2.33 from 115 to 268 kg.

Campaign S6 was increased further by a factor of 1.26 to 338 kg (Stage 3a input). The only other change was a modification to the control software to maintain adequate agitation of the product slurry during the latter stages of transfer to the centrifuge.

For both S5 and S6, the product was isolated on a centrifuge in place of the vat filter employed for S3 and S4. This resulted in more efficient liquor removal, which was reflected in improved product assay.

Comparison of Yield and Purity for Campaigns S3 to S6.

Table One gives a comparison of yield and purity data for Campaigns S3 to S6 inclusive

TABLE ONE
Stage 4 Yield and Purity Data for Campaigns S3 to S6

| | | | | | Camp'n | Stage 4 |
|---|---|---|---|---|---|---|
| | | | | Camp'n | Assay- | HPLC |
| | | Stage 3a | | Physical | based | Purity |
| | Stage 4 | Input | Stage 4 Output | Yield | Yield | (Area |
| Campaign | Batch Number | (kg) | (kg) | (%) | (%) | %) |
| S3 | 800094080 | 102.5 | 68.2 | 72.6 | Data not | 97.5 |
| | | | | | available | |
| S4 | 800109390 | 114.5 | 96.0 | 96.0 | 93.2 | 98.9 |
| S5 | 80012463A | 274.1 | 202.2 | 84.2 | 81.6 | 99.5 |
| | 80012465A | 261.2 | 210.5 | | | 99.5 |
| S6 | 80013029A, B | 343.6 | 266.2 | 87.0 | 85.2 | 98.5^{a} |
| | 80013131A, B, C | 333.8 | 273.4 | | | 97.1^{a} |

^{a}Average value of part-batches

A dramatic increase in Stage 4 physical yield was observed between Campaigns S3 and S4. The main reason for this was the modified isolation procedure, in which an additional heptane charge and vacuum distillation were incorporated. This lowered the proportion of ethyl acetate present in the supernatant liquors and thus lowered the product solubility in the liquors thereby improving the yield.

The S5 yield was disappointing compared with S4. Several possible reasons for the shortfall were identified:

Emulsified material was observed throughout the aqueous layers during the phase separations. However, examination of this material in the laboratory showed that it contained only small amounts of product.

Mass balance studies indicated that the main losses were to the mother liquors. It appears that insufficient ethyl acetate was removed during the second vacuum distillation, despite the improvements made prior to S4. It is possible that the high yield observed in S4 was due to ‘over-stripping’ as all volumes were estimated visually. In S5, volumes were measured more accurately by means of radar devices.

Some of the losses may have resulted from poor centrifugation technique. If the product slurry or washes are fed too rapidly on to the centrifuge, physical losses may occur.

On transferring the first S5 batch to the centrifuge, a significant quantity of solids remained in the crystallization vessel. Additional heptane had to be charged in order to mobilize this material and this will have had some detrimental effect on yield.

For Campaign S6, attempts were made to avoid the losses highlighted in (3) and (4) above. Particular attention was paid to operation of the centrifuge in order to avoid overflow of the basket. Agitation was maintained throughout transfer of the product slurry to the centrifuge in order to prevent solids settling out. These efforts were successful in improving the yield although not to the S4 level.

In general, the assay-based yields have been slightly lower than the physical yields reflecting the fact that the Stage 4 product generally has a lower assay than the Stage 3a input. Some variation in HPLC purity (area %) is seen across the campaigns (97.1 to 99.5 area %) although all batches have processed onward to give satisfactory purities in the downstream stages.

Process Outline

Below is the process outline of the Stage 4 process employed in Campaign S6. Process outlines for Campaigns S3, S4 and S5 are contained in the relevant campaign reports. The actual S6 charges were somewhat lower than those shown in the outline.

| Step | Description |
|---|---|
| 1. | Charge N-Methyl Indole Acid Stage 3A (370 Kg) to vessel V3166 via the solids |
| | charge chute. |
| 2. | Charge dichlorobis(triphenylphosphine)palladium(II) (3.84 Kg) to vessel V3166 |
| | via the seed pot. |
| 3. | Charge copper (I) iodide (1.06 Kg) to vessel V3166 via the seed pot. |
| 4. | Charge ethyl acetate (2048 Kg) to vessel V3166 via drums. |
| 5. | Start agitator. |
| 6. | Purge vessel V3166 three times with nitrogen. |
| 7. | Heat contents of vessel V3166 to 28 to 32° C. |
| 8. | Charge (trimethylsilyl)acetylene (122 Kg) to dispenser R3173. |
| 9. | Transfer contents of dispenser R3173 to V3166 keeping the batch temperature in |
| | the range 28 to 32° C. |
| 10. | Charge ethyl acetate (100 Kg) to dispenser R3173. |
| 11. | Transfer contents of dispenser R3173 to V3166 keeping the batch temperature in |
| | the range 28 to 32° C. |
| 12. | If necessary, adjust batch temperature to 28 to 32° C. |
| 13. | Charge triethylamine (126 Kg) to dispenser R3173. |
| 14. | Transfer contents of dispenser R3173 to V3166 keeping the batch temperature in |
| | the range 28 to 40° C. |
| 15. | Charge ethyl acetate (100 Kg) to dispenser R3173. |
| 16. | Transfer contents of dispenser R3173 to V3166 keeping the batch temperature in |
| | the range 28 to 40° C. |
| 17. | Heat contents of vessel V3166 to 38 to 42° C. |
| 18. | Stir contents of vessel V3166 at 38 to 42° C. for NLT 4 hours. |
| 19. | During stir-out charge water (2228 L) to vessel V1001 via bulk supply. |
| 20. | Start V1001 agitator. |
| 21. | Sparge contents of vessel V1001 through dip pipe with nitrogen for NLT 30 |
| | minutes. |
| 22. | Hold contents of V1001 until needed at Step 27/32 |
| 23. | Sample contents of vessel V3166 for completion (DI/SD469/401). |
| | Limit: NMT 1% Stage 3A. |
| | If complete, continue processing at Step 26. |
| | If incomplete, continue processing at Step 24. |
| 24. | Stir contents of vessel V3166 at 38 to 42° C. for NLT 2 hours. |
| 25. | Sample contents of vessel V3166 for completion (DI/SD469/401). |
| | Limit: NMT 1% Stage 3A. |
| | If complete, continue processing at Step 26. |
| | If incomplete, consult chemist. |
| 26. | Cool contents of vessel V3166 to 15 to 25° C. |
| 27. | Transfer approximately half the contents of V1001 to vessel V3166 keeping the |
| | batch temperature in the range 15 to 25° C. |
| 28. | Stir contents of vessel V3166 at 15 to 25° C. for NLT 30 minutes. |
| 29. | Allow contents of vessel V3166 to settle at 15 to 25° C. for NLT 30 minutes. |
| 30. | Separate lower, aqueous layer to receiver R1002, retaining any interfacial material |
| | with the batch layer |
| 31. | Drum up contents of receiver R1002 for disposal. |
| 32. | Transfer remaining contents of V1001 to vessel V3166 keeping the batch |
| | temperature in the range 15 to 25° C. |
| 33. | Stir contents of vessel V3166 at 15 to 25° C. for NLT 30 minutes. |
| 34. | Allow contents of vessel V3166 to settle at 15 to 25° C. for NLT 30 minutes. |
| 35. | Separate lower, aqueous layer and any interfacial material to receiver R1002. |
| 36. | Drum up contents of receiver R1002 for disposal. |
| 37. | Transfer contents of vessel V3166 to vessel V1001. |
| 38. | Vacuum-distil solvent from vessel V1001 to receiver R3350 until a residual |
| | volume of approximately 723 L is reached. |
| | Maximum batch temperature = 35° C. |
| 39. | Release vacuum to nitrogen and charge heptane (937 Kg) to vessel V1001 from |
| | drums. |
| 40. | Vacuum-distil solvent from vessel V1001 to receiver R3350 until a residual |
| | volume of approximately 1408 L is reached. |
| | Maximum batch temperature = 35° C. |
| 41. | Cool contents of vessel V1001 to 0 to −5° C. |
| 42. | Drum up contents of receiver R3350 for disposal. |
| 43. | Stir contents of vessel V1001 at 0 to −5° C. for NLT 1 hour. |
| 44. | Charge heptane (547 Kg) to header A3160. |
| 45. | Cool contents of header A3160 to 0 to −5° C. |
| 46. | Transfer approximately one quarter of the contents of vessel V1001 to peeler |
| | centrifuge D3353 taking liquors to receiver R3350. |
| 47. | Transfer approximately 186 L of heptane from header A3160 to peeler centrifuge |
| | D3353 as a cake wash, taking liquors to receiver R3350. |
| 48. | Spin dry and discharge solid to kegs. |
| 49. | Repeat steps 46-48 two more times. |
| 50. | Transfer remaining contents of vessel V1001 to peeler centrifuge D3353 taking |
| | liquors to receiver R3350. |
| 51. | Charge heptane (127 Kg) to vessel V1001. |
| 52. | Cool contents of vessel V1001 to 0 to −5° C. |
| 53. | Transfer contents of vessel V1001 to Peeler centrifuge D3353 as a cake wash, |
| | taking liquors to receiver R3350. |
| 54. | Spin dry and discharge solid to kegs. |
| 55. | Drum up contents of receiver R3350 for disposal. |
| 56. | Charge wet-cake to dryer K3045. |
| 57. | Dry wet-cake in dryer K3045 at 25 to 35° C. until an LOD of NMT 1.0% is |
| | achieved. |
| 58. | Discharge product from dryer K3045 to drums. |

Expected dry weight = 285 kg (84%)

Equipment Train

For Campaigns S3 and S4, Stage 4 aqueous work-up and crystallisation were performed in PV351, a 300-gallon glass-lined vessel. The product was isolated in PD351, a ceramic vat-filter associated with PV351 and dried in PK601, a single cabinet air tray-dryer.

Campaigns S5 and S6 were carried out in V3166, a 1000-gallon hastelloy reactor. Crystallisation was performed in V1001, a 1000-gallon glass-lined vessel and the product was isolated on D3353, a hastelloy peeler centrifuge. Drying took place in K3045, a stainless steel vacuum tray dryer.

Quality-Critical Parameters

No laboratory work to identify quality-critical parameters has been performed for Stage 4. The product manufactured in Campaigns S3 to S6 has consistently met specification and no quality-critical parameters have been identified from the processing records.

The main quality issue for Stage 4 has been the residual level of heavy metals (palladium and copper) in the isolated product. A substantial amount of work has been directed towards reducing the levels of these metals in the Stage 4 product and thus in subsequent stages. This work is described in detail in a separate report. The ‘low-palladium’ Stage 4 to 7 process has now been successfully demonstrated in the Pilot Plant on approximately 10% of the full manufacturing scale

Parameters Affecting Yield

Four main factors affecting yield have been identified based on processing observations:

During the work-up in Campaigns S5 and S6, significant amounts of emulsified material were found throughout the aqueous layer. This material was sampled and found to contain very little product. However, for the sake of process robustness, it was decided to evaluate filtering the two-phase mixture prior to separating the layers. This was demonstrated in the recent Pilot Campaign and gave rise to very clean separations.

The Stage 4 product is crystallised by performing a solvent-exchange from ethyl acetate to heptane. Residual ethyl acetate solubilises the product resulting in loss of yield to the mother liquors. Varying degrees of efficiency in removing ethyl acetate are thought to be responsible for some of the yield variations observed in recent campaigns. In order to ensure consistently low levels of ethyl acetate, the effect of an additional heptane charge and vacuum distillation have been investigated. These modifications have consistently given physical yields in excess of 90% at 10-L scale and in the recent Pilot Campaign.

In the first batch of Campaign S5, a significant quantity of Stage 4 product remained in the crystalliser after transfer of the batch to the centrifuge. This was because the process-control software stopped agitation when the slurry reached a low volume in order to prevent splashing. The software has since been modified to continue stirring throughout the transfer, thereby preventing solids from settling out.

Centrifuge technique is an important factor in preventing yield loss at each stage of the process. While none of the losses at Stage 4 have been directly attributed to poor centrifugation, it is important to ensure that the slurry and washes are applied at the correct rate in order to prevent product from spilling over the sides of the basket.

Stage 4 Reaction Completion

Conversion of Stage 3a to Stage 4 is monitored by HPLC analysis of the reaction mixture. Table Two gives a comparison of final in-process levels of Stage 3a and the Stage 3a content of the isolated Stage 4 product. The current in-process limit for unreacted Stage 3a is 1.0 area % and this has remained unchanged since Campaign S2. For Campaigns S3 to S6 inclusive, there was no intermediate specification for residual Stage 3a. After S6, a specification of ‘NMT 1.0 area %’ was introduced, based on the historical data shown in Table Two. All batches from S3 to S6 satisfied the 1.0% limit with ease. However, the data in Table Two indicate that the in-process analysis underestimates the level of residual Stage 3a relative to the intermediate analysis. This effect was even more pronounced in the Pilot Campaign (batch 800141090). Here, the final in-process analysis showed no Stage 3a detected while one part-batch of the isolated product contained 1.16 area % and thereby failed to meet the specification. No satisfactory explanation has yet been put forward for this discrepancy.

For safety reasons, the Pilot batch was cooled to −5 to 0° C. and the agitator was stopped before sampling. This contrasts with the earlier batches, where sampling was performed at the reaction temperature (38 to 42° C.) with agitation. Unreacted Stage 3a may have precipitated on cooling and settled to the base of the vessel in the absence of agitation. If this were the case, sampling the batch solution would not have given a true reflection of the Stage 3a level.

The initial analysis performed on the Pilot batch 800141090 showed Stage 3a levels up to around 6 area %. Moreover, the apparent levels of Stage 3a in a particular Stage 4 sample increased with time while the sample was stored as an acetonitrile solution in a glass vial. No similar increase was observed when the samples were prepared in PTFE vials. After an extensive analytical investigation, it was concluded that Stage 4 in acetonitrile degrades in glass vials to give an impurity that co-elutes with the Stage 3a product. (It is highly improbable, from a chemical viewpoint, that the Stage 4 product actually reverts to Stage 3a). However, this sample degradation does not account for the apparent high level of Stage 3a recorded for batch 800141090 (1.16 area %) since this value was obtained using PTFE vials.

The intermediate analysis of S6 batches 80013029A,B and 80013031A,B,C show high levels (up to 1%) of an impurity which is distinct from Stage 3a but runs close to it, separated by about 0.1 minutes. It is possible that more than one impurity runs in this region. However, this still does not explain why the S6 completion analysis does not show any significant peaks in this region.

Further work is clearly necessary to establish the reason(s) for the discrepancy between the intermediate and in-process residual Stage 3a levels. At present, the in-process test does not appear to be a reliable predictor of the Stage 3a level in the isolated product.

TABLE TWO
Comparison of In-process and Intermediate Stage 3a levels for
Stage 4 Campaigns S3 to S6

| | | Final In-process | Stage 3a in Isolated |
|---|---|---|---|
| | | Stage 3a | Stage 4 |
| Campaign | Batch | (HPLC Area %) | (HPLC Area %) |
| S3 | 800094080 | 0.2 | 0.3 |
| S4 | 800109390 | 0.06 | 0.09 |
| S5 | 80012463A | 0.1 | 0.39 |
| | 80012465A | 0.2 | 0.56 |
| S6 | 80013029A, B | ND | 0.12 |
| | 80013031A, B, C | ND | 0.39 |

TABLE THREE
Comparison of In-process and Intermediate State 4
LOD Values for Campaigns S4 to S6

| | | Final In- | |
|---|---|---|---|
| | | process | Intermediate |
| | | LOD (% | LOD (% |
| Campaign | Batch | w/w) | w/w) |
| S3 | 800094080 | 0.1 | 0.9 |
| S4 | 800109390 | 0.3 | 0.1 |
| S5 | 80012463A | 0.04 | 0.02 |
| | 80012465A | ND | ND |
| S6 | 80013029A | 0.2 | 0.07 |
| | 80013029B | 0.1 | 0.06 |
| | 80013031A | 0.1 | 0.06 |
| | 80013031B | 0.2 | 0.06 |
| | 80013031C | ND | 0.06 |

Stage 3a

Residual Stage 3a runs between RRT 0.69 and 0.73. The data in Table Four show some variation in RRT values for this impurity. For this reason, it would be worthwhile re-analysing the retained S3 to S6 samples using the same HPLC equipment on the same day. Levels of Stage 3a recorded to date range from 0.1 to 0.56 area %. However, the recent Pilot Campaign yielded Stage 4 containing up to 1.16 area % Stage 3a. This failed the current specification (NMT 1.0% Stage 3a), but was usage-tested to give Stages 5 and 7 with excellent purity and satisfying all specifications. It would therefore appear that the current specification is too tight and that it should be revised upwards, possibly with the aid of spiking experiments. The data in Table Four indicate that more than one impurity is found in the region RRT 0.69 to 0.73. One of these impurities apparently increases when Stage 4 samples are stored in acetonitrile in glass vials. Further work is required to determine the nature of this impurity and to achieve better separation from residual Stage 3a.

Stage 4 Regioisomer

The Stage 4 regioisomer is derived from the product of iodination at the 3-position, formed during the Stage 3 reaction. An authentic sample of the impurity has been synthesised and used as a reference standard to quantify levels in recent batches. Stage 4 batches from Campaigns S5 and S6 were analysed and the regioisomer was not detected at levels higher than 0.1 area %. Although the Stage 3 reaction generates up to 25% of the regioisomeric iodo compound, it is largely removed during the isolation from DMF/water and the subsequent isopropyl alcohol centrifuge washes. In one batch of Campaign S6, a high level of the regioisomer was found in the isolated Stage 3 product and this was attributed to inefficient washing on the centrifuge. The impurity was readily removed at Stage 3a by employing additional methanol centrifuge washes during the isolation. Clearly, efficient centrifugation and washing at Stage 3 are vital to achieving good control over this impurity. Levels of Stage 4 regioisomer in excess of 0.1 area % have not been carried forward to Stage 5. It is therefore not possible to set a meaningful specification for this impurity at the present time.

TABLE FOUR
Stage 4 HPLC Profiles for Campaigns S3 to S6

| | | RRT | | | | | | | | |
|---|---|---|---|---|---|---|---|---|---|---|
| Campaign | Batch | 0.13 | 0.69 | | 0.70 | 0.71 | 0.73 | 0.85 | 1.00 | 1.17 |
| S3 | 800094080 | 0.49 | ND | | 0.34 | 0.32 | ND | ND | 97.49 | 1.20 |
| S4 | 800109390 | ND | ND | | ND | 0.1 | 0.61 | ND | 98.69 | ND |
| S5 | 80012463A | ND | 0.39 | | ND | ND | ND | ND | 99.56 | ND |
| | 80012465A | ND | 0.56 | | ND | ND | ND | 0.39 | 99.05 | ND |
| S6 | 80013029A | ND | ND | ND | ND | ND | ND | ND | 98.5 | ND |
| | 80013029B | ND | ND | ND | ND | ND | ND | ND | 98.5 | ND |
| | 80013031A | ND | 0.7 | 0.5 | ND | ND | ND | ND | 97.2 | ND |
| | 80013031B | ND | 1.0 | 0.4 | ND | ND | ND | ND | 96.7 | ND |
| | 80013031C | ND | 0.5 | 0.3 | ND | ND | ND | ND | 97.3 | ND |

| | |
|---|---|
| Current specification | NLT 94 |

### EXAMPLE 6

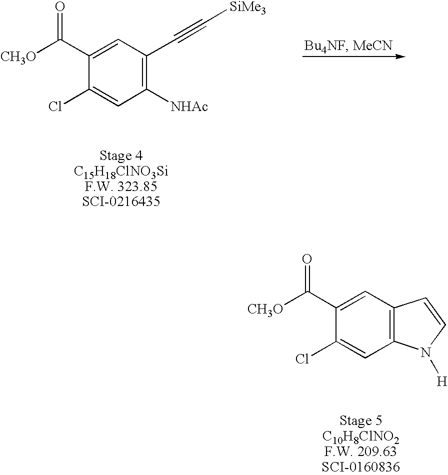

Prior to Campaign S3, tetrabutylammonium fluoride (TBAF) and Stage 4 were charged to the reactor as solids before adding solvent. In Campaign S2, this led to an exothermic reaction between the solids with potential for serious safety consequences. For Campaign S3, TBAF was added successfully as a THF solution.

In Campaign S4, Stage 5 was isolated for the first time as a solid to allow the possibility of registering it as a ‘cGMP Starting Material’. The reaction solvent was changed from THF to acetonitrile and the product was isolated by distilling off excess solvent and adding to a large volume of water. The modified process worked well and yielded Stage 5 product with satisfactory quality.

In Campaign S5, the batch size (Stage 4 input) was increased by a factor of 2.18 and the initial acetonitrile charge was increased from 1.9 to 5.5 volumes to compensate for the very high minimum stirring volume in the reaction vessel. The excess acetonitrile was removed by vacuum distillation prior to quenching the reaction mixture.

The process employed in Campaign S6 was virtually unchanged from S5 apart from a 31% increase in batch size. This increase permitted a smaller volume of acetonitrile to be used for the reaction. However, due to volume constraints it was necessary to quench the batch in two halves.

An additional change introduced for both S5 and S6 was the isolation of the product on a centrifuge in place of a vat filter. This generally resulted in better de-liquoring of the wet-cake leading to improved purity and shorter drying times.

Comparison of Yield and Purity for Campaigns S3 to S6.

TABLE ONE
Stage 5 Yield and Purity Data for Campaigns S4 to S6

| | | | | | Campaign | Stage 5 |
|---|---|---|---|---|---|---|
| | | | | Campaign | Assay- | HPLC |
| | | | | Physical | based | Purity |
| | Stage 5 Batch | Stage 4 Input | Stage 5 Output | Yield | Yield | (Area |
| Campaign | Number | (kg) | (kg) | (%) | (%) | %) |
| S4 | 80011358A, B | 99.0 | 58.2 | 90.8 | 83.2 | 94.7 |
| S5 | 800125330 | 202.2 | 115.4 | 90.6 | 89.1 | 96.4 |
| | 800125780 | 210.5 | 126.6 | | | 96.3 |
| S6 | 80013075A, B | 266.2 | 157.2 | 92.0 | 91.4 | 95.4 |
| | 80013076A, B | 273.4 | 164.2 | | | 97.0 |

The data show that physical yield has remained virtually unchanged since Campaign S4. However, Campaigns S5 and S6 showed a dramatic improvement in assay-based yield. The reasons for this are not fully understood. Certainly, the material isolated in S5 and S6 had a much higher assay (>93% w/w) than the S4 material (84.8% w/w). This was attributed to isolation on a centrifuge, giving more efficient liquor removal and thus reduced retention of impurities. Similarly, the Stage 4 input for S5 and S6 was purer (>93.8% w/w) than that used in S4. These facts in themselves do not explain the dramatic increase in assay-based yield. However, it is possible that a purer Stage 4 input may have resulted in a better crystallization and thus a more efficient product recovery.

The Stage 5 product generated in Campaigns S5 and S6 had a higher area % purity than that from S5. Also, as noted above, the % w/w assay was much higher for the S5 and S6 material. Both these observations are consistent with the use of centrifugation in S5 and S6 and the consequent improved efficiency of liquor removal. In S4, there was a marked discrepancy between the product assay (84.8% w/w) and the HPLC purity (94.7 area %). This is thought to be due to the retention of TBAF-related residues, which are not UV-active. Much better agreement between the assay and area % values was observed in S5 and S6.

Process Outline

Below is the process outline of the Stage 5 process employed in Campaign S6.

| Step | Description |
|---|---|
| 1. | Preliminary Operation |
| | Preparation of 34% w/w TBAF/Acetonitrile solution |
| | Charge tetrabutylammonium fluoride trihydrate (372 Kg) to vessel V1 via the |
| | manway. |
| 2. | Charge acetonitrile (720 Kg) to vessel V1 via drums. |
| 3. | Stir contents of vessel V1 at 15 to 25° C. for at least 1 |
| | hour. |
| 4. | Hold contents of vessel V1 for use at Step 9. |
| 5. | Main Process |
| | Charge N-methyl indole acid Stage Four (305 Kg) to vessel V3166 via the solids |
| | charge chute. |
| 6. | Charge acetonitrile (1168 Kg) to vessel V3166 via drums. |
| 7. | Start agitator on V3166. |
| 8. | Adjust contents of vessel V3166 to 20 to 30° C. |
| 9. | Transfer the tetrabutylammonium fluoride trihydrate solution from vessel V1 to |
| | vessel V3166 maintaining batch temperature in range 20 to 35° C. |
| 10. | Charge acetonitrile (49 Kg) to vessel V1 via drums. |
| 11. | Transfer contents of V1 to vessel V3166 as a line rinse. |
| 12. | Heat contents of vessel V3166 to reflux (78-80° C.). |
| 13. | Stir contents of vessel V3166 at reflux for NLT 7 hours. |
| 14. | During the reflux period, charge to V1001 water (1909 L) via direct line. |
| 15. | Cool contents of vessel V3166 to 20-30° C. and sample for completion (DI/SD- |
| | 469/501). |
| | Limit: Stage 4 NMT 1% & Stage 5 NLT 93% |
| | If complete, continue processing at Step 18. |
| | If incomplete, continue processing at Step 16. |
| 16. | Re-heat contents of vessel V3166 to reflux and stir for NLT 2 hours. |
| 17. | Cool contents of vessel V3166 to 20-30° C. and sample for completion (DI/SD- |
| | 469/501). |
| | Limit: Stage 4 NMT 1% & Stage 5 NLT 93% |
| | If complete, continue processing at Step 18. |
| | If incomplete, consult chemist. |
| 18. | Set lines on V3166 for vacuum distillation to receiver R1002 & distil solvent to a |
| | batch volume of 1200 L, maintaining the contents temperature below 35° C. |
| 19. | Drum up the contents of receiver R1002. |
| 20. | Transfer half of contents of vessel V3166 to vessel V1001, over NLT 80 minutes, |
| | maintaining temperature in V1001 between 15 & 25° C. |
| 21. | Stir contents of V1001 at 15 to 25° C. for NLT 1 hour. |
| 22. | Charge water (900 L) to header A3160. |
| 23. | Transfer approximately half the contents of V1001 to Peeler centrifuge D3353, |
| 23. | taking liquors to receiver R3350. |
| 24. | Transfer water (198 L) from header A3160 to Peeler centrifuge D3353 as cake |
| | wash, taking liquors to R3350 |
| 25. | Spin dry and discharge solid to kegs. |
| 26. | Transfer remaining contents of V1001 to Peeler centrifuge D3353, taking liquors |
| | to receiver R3350. |
| 27. | Charge to vessel V1001 water (198 L) and stir. |
| 28. | Transfer contents of vessel V1001 to Peeler centrifuge D3353 as cake wash, taking |
| | liquors to R3350 |
| 29. | Spin dry and discharge solid to kegs. |
| 30. | Drum up the contents of R3350 for disposal. |
| 31. | Charge water (1801 L) to V1001 via direct line. |
| 32. | Transfer remaining contents of vessel V3166 to vessel V1001, over NLT 80 |
| | minutes, maintaining temperature in V1001 between 15 & 25° C. |
| 33. | Charge water (108 L) to V3166. |
| 34. | Transfer contents of V3166 to V1001 while maintaining the contents of V1001 at |
| | 15 to 25° C. |
| 35. | Stir contents of V1001 at 15 to 25° C. for NLT 1 hour. |
| 36. | Transfer approximately half the contents of V1001 to Peeler centrifuge D3353, |
| | taking liquors to receiver R3350. |
| 37. | Transfer water (198 L) from header A3160 to Peeler centrifuge D3353 as cake |
| | wash, taking liquors to R3350 |
| 38. | Spin dry and discharge solid to kegs. |
| 39. | Transfer remaining contents of V1001 to Peeler centrifuge D3353, taking liquors |
| | to receiver R3350. |
| 40. | Charge to vessel V1001 water (198 L) and stir. |
| 41. | Transfer contents of vessel V1001 to Peeler centrifuge D3353 as cake wash, taking |
| | liquors to R3350 |
| 42. | Spin dry and discharge solid to kegs. |
| 43. | Transfer any residual water from A3160 to R3350. |
| 44. | Drum up the contents of R3350 for disposal. |
| 45. | Charge wet-cake to dryer K3045. |
| 46. | Dry product at 35 to 40° C. until LOD of NMT 0.5% w/w is achieved. |
| 47. | Discharge product from dryer K3045 to drums. |

Expected dry weight = 179 kg (91%)

Equipment Train

In Campaign S4, the Stage 5 reaction and aqueous quench were carried out in PV351, a 300-gallon glass-lined vessel in a Pilot Plant. The product was filtered on PD351, a ceramic filter associated with PV351 and dried in PK601, a single cabinet air tray-dryer.

Stage 5 manufacture chemistry took place in V3166, a 1000-gallon hastelloy reactor and the aqueous quench was performed in V1001, a 1000-gallon glass-lined vessel. The product was isolated on the hastelloy Peeler centrifuge D3353 and dried in K3045, a stainless steel vacuum tray-dryer.

Parameters Affecting Yield

Laboratory experiments have shown that the Stage 5 yield is affected by:

The concentration of the acetonitrile product solution prior to quenching;

The volume of water employed in the quench.

It was shown that reducing the volume of the acetonitrile solution by 25% (i.e. distilling out more acetonitrile) increased the physical yield from 91.8 to 93.6% and reduced the purity from 97.55 to 97.25 area %. Reducing the acetonitrile solution volume by 25% and increasing the water quench volume by 25% increased the yield further to 97.3% and reduced the purity to 97.02 area %. It would therefore appear that a significant yield improvement can be achieved, while maintaining satisfactory quality.

Stage 5 Reaction Completion

Conversion of Stage 4 to Stage 5 is monitored by HPLC analysis of the reaction mixture. Stage 4 is not converted to Stage 5 directly, but proceeds via at least one intermediate species.

TABLE TWO
Comparison of In-process and Intermediate
HPLC Analysis for Campaigns S4 to S6

| | | In- | Inter- | In- | |
|---|---|---|---|---|---|
| | | process | mediate | process | Intermediate |
| | | Stage 4 | Stage 4 | Stage 5 | Stage 5 |
| | | (Area | (Area | (Area | (Area |
| Campaign | Batch | %) | %) | %) | %) |
| S4 | 80011358A, B | ND | ND | 95.0 | 94.7 |
| S5 | 800125330 | 1.0 | ND | 98.0 | 96.4 |
| | 800125780 | 1.0 | ND | 94.0 | 96.3 |
| S6 | 80013075A, B | ND | 0.04 | 99.0 | 95.4 |
| | 80013076A, B | ND | ND | 98.0 | 97.0 |

The in-process and intermediate analysis is compared for Campaigns S4 to S6 in Table Two. Both S5 batches were right on the in-process limit, but gave product with no detectable Stage 4. The reaction period was increased from S5 (6 hours) to S6 (7 hours) thus ensuring complete consumption of Stage 4.

Stage 5 HPLC Profile

Table Four shows the Stage 5 HPLC profiles for Campaigns S4 to S6 inclusive. It is difficult to make direct comparisons between the profiles as the analysis of the various batches was carried out at different times using different equipment. There will therefore inevitably be some variation in RRT values from one campaign to the next.

With the exception of the Stage 4 product, none of the impurities in Stage 5 have been identified. However, a few general observations can be made:

The S4 and S5 batches contained relatively high levels of the early-running impurities (RRT 0.97 and 0.98) but these were absent from the S6 batches.

Very high levels (1.98 and 0.84 area %) of the RRT 1.03 and 1.04 impurities were detected in S6 batch 800130750, whereas the levels in batch 800130760 were much lower and these impurities were not detected at all in S4 and S5.

The 4-chloro regioisomer of Stage 5 is derived from the regioisomeric iodination product generated at Stage 3. An authentic sample of this impurity has been prepared. However, it co-elutes with Stage 5 so it is not possible to quantify levels in the plant batches. The precursor Stage 4 regioisomer has been shown to be consistently below 0.1 area %, so it is extremely unlikely that significant levels will be detected at Stage 5.

TABLE FOUR
Stage 5 HPLC Profiles for Campaigns S4 to S6

| | Component (Area %) | | | | | | | | | | | | |
|---|---|---|---|---|---|---|---|---|---|---|---|---|---|
| | | | | | | | | | | | RRT | | |
| Batch | | RRT | RRT | RRT | RRT | RRT | RRT | RRT | RRT | RRT | 1.40 | RRT | RRT |
| Number | St 5 | 1.01 | 1.03 | 1.04 | 1.20 | 1.22 | 1.25 | 1.26 | 1.31 | 1.38 | (St4) | 1.60 | 1.64 |
| 80011416A^{a} | 94.71 | 0.8 | ND | ND | 1.01 | ND | ND | 0.79 | 0.60 | ND | ND | ND | ND |
| 80011416B^{a} | 94.56 | 0.82 | ND | ND | 0.99 | ND | ND | 0.78 | 0.61 | ND | ND | ND | ND |
| 800125330 | 96.4 | ND | 0.15 | ND | ND | 0.52 | ND | ND | ND | ND | ND | 0.18 | ND |
| 800125780 | 96.3 | ND | ND | ND | ND | 0.62 | ND | ND | ND | ND | ND | 0.19 | ND |
| 80013075^{b} | 95.4 | ND | 1.98 | 0.84 | ND | 0.51 | 0.3 | 0.15 | ND | 0.1 | 0.04 | ND | 0.43 |
| 80013076^{b} | 96.98 | ND | 0.7 | 0.2 | ND | 0.7 | 0.4 | <0.1 | ND | 0.2 | ND | ND | 0.2 |

^{a}Re-dried batch 80011358A and B
^{b}Values shown are average for part-batches A and B

The foregoing examples are illustrative only, and are not to be viewed as limitations on the scope of the invention.

1. A method to synthesize an indole compound, said method comprising:
(1) providing a para-amino benzoate derivative of formula (II): 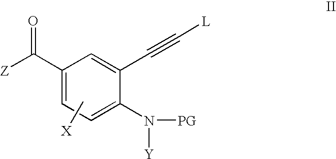
and
(2) cyclizing the acetylenic substituent onto the amino group to form an indole compound of formula (III): 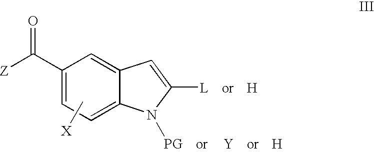
wherein:
PG is a protecting group;
X represents 0-3 substituents selected from halo, CF₃, C1-C6 alkyl, and C1-C6 alkoxy;
Y is H or optionally substituted C1-C6 alkyl or optionally substituted C1-C6 sulfonyl, or Y can be a protecting group that may be the same as or different from PG; and
Z is OR or NR₂ or a 5-6 membered azacyclic group that forms an amide with the benzoate carbonyl; and
L is an optionally substituted C1-C6 alkyl or R₃Si— wherein each R is independently C1-C6 alkyl or phenyl.

2. The method of claim 1, wherein X represents at least one substituent,
and wherein at least one substituent represented by X is ortho to the —C(O)Z group.

3. The method of claim 2, wherein X represents exactly one substituent, which substituent is chloro, methoxy, or methyl.

4. The method of claim 3, wherein X represents chloro, and PG is acetyl or formyl, and wherein the cyclized product of formula (III) has H at N-1 of the indole.

5. The method of claim 1, wherein the compound of formula (II) is prepared by acetylenation of a compound of formula (I): 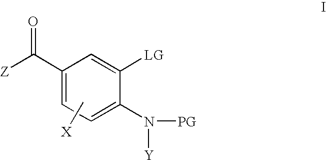
wherein LG represents an Iodo group (—I) or another leaving group.

6. The method of claim 5, wherein L in the compound of formula (II) is a trialkylsilyl group,
and the step of cyclizing the acetylenic substituent onto the amino group is accomplished by treating the compound of formula (II) with a fluoride source in an organic solvent.

7. The method of claim 5, wherein the fluoride source is a tetra-alkyl ammonium fluoride and the product of formula (III) has H at C-2 of the indole.

8. The method of claim 1, wherein the protecting group PG is an optionally substituted C1-C6 acyl group and the product of formula (III) has H at N-1 of the indole.

9. The method of claim 5, wherein the compound of formula (I) is of the following formula: 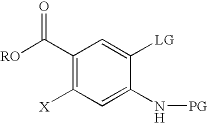
wherein R represents optionally substituted C1-C6 alkyl or optionally substituted phenyl;
X represents Cl, Me or OMe;
LG is a leaving group; and
PG is a protecting group.

10. The method of claim 9, wherein PG is acetyl and the product of formula (III) has H at N-1 of the indole.

11. The method of claim 10, wherein L in the compound of formula (II) is trimethylsilyl.

12. The method of claim 11, wherein LG is —I, and the product is an indole of the following formula:

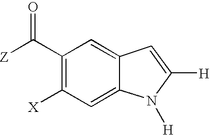

13. The method of claim 12, wherein Z is C1-C6 alkoxy and X is Cl.

14. A method to synthesize an indole of formula (VI) by reacting a compound of formula (IV) and at least a catalytic amount of a compound of formula (V) in the presence of fluoride ion in an organic solvent: 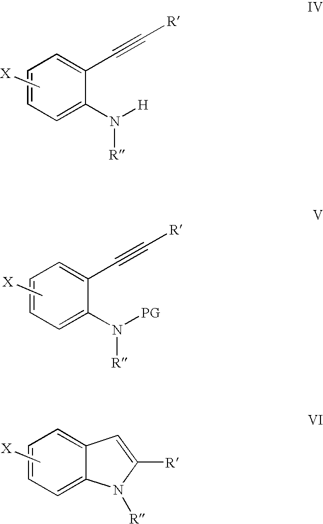
wherein:
X represents 0-3 substituents suitable for an aryl group;
PG represents an optionally substituted C1-C6 acyl group;
each R′ independently represents H, optionally substituted C1-C6 alkyl, optionally substituted C5-C12 aryl including heteroaryl groups containing 1-3 heteroatoms selected from N, O and S, optionally substituted C6-C13 arylalkyl, or R₃Si wherein each R is independently C1-C6 alkyl or phenyl;
each R″ independently represents H, optionally substituted C1-C6 alkyl, optionally substituted C5-C12 aryl including heteroaryl groups containing 1-3 heteroatoms selected from N, O and S, optionally substituted C6-C13 arylalkyl, an optionally substituted C1-C6 alkylsulfonyl group, an optionally substituted C5-C12 arylsulfonyl group, an optionally substituted C1-C6 alkoxycarbonyl group, or an optionally substituted C1-C6 acyl group.

15. The method of claim 14, wherein R′ in the compounds of formula (IV) and (V) is R₃Si, wherein each R is independently C1-C6 alkyl or phenyl,
and R′ is H in the product of formula (VI).

16. The method of claim 14, wherein each R″ is H.

17. A compound of formula (I): 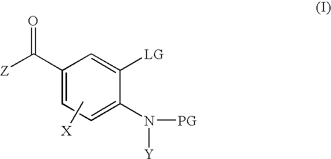
wherein:
LG is a leaving group;
PG is a protecting group;
X represents 0-3 substituents selected from halo, CF₃, C1-C6 alkyl, and C1-C6 alkoxy;
Y is H or optionally substituted C1-C6 alkyl or optionally substituted C1-C6 sulfonyl, or Y can be the same as PG; and
Z is OR or NR₂ or a 5-6 membered azacyclic group that forms an amide with the benzoate carbonyl.

18. A compound of formula (II): 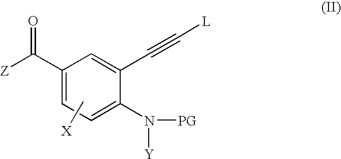
wherein:
PG is a protecting group;
Z is OR or NR₂ or a 5-6 membered azacyclic group that forms an amide with the benzoate carbonyl;
L is an optionally substituted C1-C6 alkyl group or R₃Si;
X represents 0-3 substituents selected from halo, CF₃, C1-C6 alkyl, and C1-C6 alkoxy;
Y is H or optionally substituted C1-C6 alkyl or optionally substituted C1-C6 sulfonyl, or Y can be the same as PG; and
and each R is independently C1-C6 alkyl or phenyl.
